(19) United States
(12) Patent Application Publication
(10) Pub. No.: US 2008/0227846 A1
(43) Pub. Date: Sep. 18, 2008

(54) METHODS OF TREATING JUVENILE TYPE 1 DIABETES MELLITUS

(75) Inventors: Inderjit Singh, Mt. Pleasant, SC (US); Lyndon Key, Hollywood, SC (US)

Correspondence Address: FULBRIGHT & JAWORSKI L.L.P., 600 CONGRESS AVE., SUITE 2400, AUSTIN, TX, 78701, US

(73) Assignees: MUSC Foundation for Research Development, Charleston, SC (US); MEDICAL UNIVERSITY OF SOUTH CAROLINA

(21) Appl. No.: 12/047,871

(22) Filed: Mar. 13, 2008

Related U.S. Application Data
(60) Provisional application No. 60/894,594, filed on Mar. 13, 2007.

(51) Int. Cl. A61K 31/404; A61K 31/366; A61P 3/10; A61K 31/40

(52) U.S. Cl. 514/419; 514/460; 514/423

(57) ABSTRACT

The present disclosure describes methods for treating or preventing Type 1 diabetes mellitus in juveniles, particularly in juveniles newly diagnosed with Type 1 diabetes. This prevention or treatment of Type 1 diabetes is achieved by administering one or more therapeutic agents to a juvenile in need, wherein the therapeutic agent is, for example, a competitive inhibitor of mevalonate synthesis, a competitive inhibitor of 3-hydroxy-3-methylglutaryl coenzyme A (HMG-CoA) reductase, or an inducer of AMP protein kinase (AMPK) activity. In certain embodiments, juveniles with Type 1 diabetes are treated with an HMG-CoA reductase inhibitor such as a statin, thereby decreasing the destruction of islet cells, or maintaining endogenous insulin production, in the juvenile.

### CROSS-REFERENCES TO RELATED APPLICATIONS

This application claims benefit of priority to U.S. Provisional No. 60/894,594, filed Mar. 13, 2007, and International Patent Application PCT/______, entitled “Methods of Treating Juvenile Type 1 Diabetes Mellitus,” filed on Mar. 13, 2008, both of which are incorporated herein by reference in their entirety.

### STATEMENT REGARDING FEDERALLY SPONSORED RESEARCH OR DEVELOPMENT

The government may owns rights in this invention pursuant to grant number FD-R003340-01 from the Food and Drug Administration.

### BACKGROUND OF THE INVENTION

1. Field of the Invention

The present disclosure relates to the treatment and prevention of type 1 diabetes mellitus in juveniles (also referred to as juvenile diabetes), for example, by suppressing or inhibiting inflammatory mediators (e.g., cytokines and inducible nitric oxide synthase) by inhibitors of the mevalonate pathway and activators of AMP-activated protein kinase. In an embodiment of the present disclosure, a juvenile with type 1 diabetes mellitus is treated with a statin.

2. Description of Related Art

In type 1 diabetes mellitus (T1DM), beta-cells in the pancreatic islets of Langerhans (“islet cells” or “beta cells”) are progressively lost, which leads to a lack of insulin, a protein hormone critical for glucose metabolism. Beta cells can compensate for the loss of a significant portion of their population, so hyperglycemia usually does not ensue until nearly all of the cells are destroyed. T1DM is estimated to account for 5-10% of all new cases of diabetes each year, and 1 to 1.5 million people are believed to be affected with this disease in the United States (Harris M., “Definition and Classification of Diabetes Mellitus and the New Criteria for Diagnosis” Chapter 32, Page 327. Diabetes Mellitus A Fundamental and Clinical Text, 2nd Edition. Ed: LeRoith D, Taylor S, Olefsky J. Lipincott Williams & Wilkins, 2000). Recently, a multicenter, CDC/NIH sponsored study (the SEARCH for Diabetes in Youth) compiled sufficient data to allow an accurate estimate of the age and race/ethnicity-specific incidence of T1DM in children less than 20. In this study, a diagnosis of T1DM was defined by the presence of diabetes auto-antibodies and insulinopenia using C-peptide levels. Recently presented data indicate that the incidence of T1DM in the U.S. is approximately 16,000 cases per year in individuals under the age of 19, with approximately 8,700 new cases occurring in the 10-19 year age group.

T1DM is a chronic autoimmune condition in which pancreatic beta cells are destroyed, resulting in dependence on exogenous insulin for life (Atkinson and Maclaren, N Engl J. Med. 331(21):1428-1436, 1994). Despite considerable progress over the past several decades (Willi and Parker, J South Carolina Med Assoc. 93:308-12, 1997), T1DM management is not optimal, as patients require multiple daily insulin injections or use of an insulin pump to avert long-term complications. Presently, frequent blood glucose monitoring and constant adjustment of insulin regimen is required to achieve optimal blood glucose levels (The Diabetes Control and Complications Trial Research Group, N Engl J Med. 329:977-986, 1993). Such strict glycemic control rarely can be achieved with current T1DM management, and overly aggressive therapy can result in recurrent severe hypoglycemia. At this time it is not possible to fully mimic the function of beta cells, and there are no established treatments that can prevent the immunological destruction of these cells in T1DM patients. Therefore, patients with this disease rely on frequent insulin injections or insulin-pump therapy to prevent acute and chronic complications, as well as premature death.

Ongoing studies are evaluating the use of genotype, auto-antibodies, and metabolic markers to screen first-degree relatives of T1DM patients who may be at risk for the disease (Diabetes Prevention Trial—Type 1 Study Group. Effects of insulin in relatives of patients with type 1 diabetes. NUM 346:1658-91, 2002; Mrena et al., J Clin Endocrinol Metab. 88(6):2682-2689, 2003). Unfortunately, it is not currently possible to identify the majority of subjects who will develop T1DM in the general population in the subclinical phase of the disease. At present, interventions that are effective at the time of clinical presentation represent the most useful form of therapy. T1DM is treated with daily injections of insulin.

Often after clinical presentation, affected individuals enter a remission phase, during which they are still able to make substantial amounts of insulin (Steele et al., Diabetes 53(2):426-433, 2004; O'Meara et al., Diabetes Care. 18(4):568-571, 1995). This period may be referred to as the “honeymoon period.” During the “honeymoon period”, which often occurs after a patient begins insulin injections, there is some restoration of insulin production, and the blood sugar levels improve to normal, or near-normal, levels. During the honeymoon period, the remaining beta cells continue to produce insulin. It can be very important to continue insulin therapy during the honeymoon period, because even low doses of insulin appear to help prolong the duration of the honeymoon period. Unfortunately, this diabetes honeymoon usually only lasts for weeks, months, or occasionally, years. Endogenous insulin secretion continues to deteriorate, usually over the first 1-2 years of disease, eventually becoming undetectable and necessitating complete reliance on exogenous insulin.

Studies of humans with T1DM and non-obese diabetic (NOD) mice (an animal model for T1DM) indicate that T1DM is a T-cell mediated inflammatory disease. In mice, lymphocytic infiltration surrounding the islet cells, termed insulitis, is the initial pathological finding, occurring at 5-8 weeks. These infiltrates contain many types of inflammatory cells including antigen-presenting cells (e.g., macrophages), T-helper cells, cytotoxic T-cell, B-lymphocytes and natural killer cells (Paintlia et al., J Neuro Sci Res. 77:63-81, 2004; Donath et al., J Mol Med. 81:455-470, 2003; Durinovic-Bello et al., Ann NY Acad Sci. 1005:288-94, 2003; Herold K C, Endocrinol Metab Clin North Am. 33(1):93-111, 2004; Faresjo et al., Scand. J Immunol. 59:517-26, 2004; Gottlieb and Hayward, Endocrinol Metab Clin North Am. 31(1):477-95, 2002; Roep B O, Diabetologia 46(3): 305-21, 2003; Matteucci et al., Clin Exp Immune 136:549-554, 2004). In NOD mice, the role of Th1-cells and inflammatory mediators produced by them (particularly IFN-γ, IL-1β and TNFα) in insulitis-induced-beta-cell apoptosis is well established. A number of recent reports also suggest that Th1-cells and their inflammatory mediators have a significant role in the pathology of T1DM in humans.

Previous attempts to preserve islet-cell-function in patients with T1DM have focused on treating patients with new-onset T1DM with immunomodulatory drugs such as prednisone, azothioprine, cyclosporine or CD3 depleting immunoglobulins (Silverstein et al., NEJM 319(10):599-604, 1988; Cook et al., Diabetes 38(6):779-783, 1989; Skyler and Rabinovitch, Journal of Diabetes & its Complications 6:77-88, 1992; Herold et al., New Eng J Med. 346:1692-1698, 2002). Each of these agents were shown to induce transient improvement in controlling diabetes and increase the remission rate when instituted soon after diagnosis. The improved metabolic control resulting from preservation of even suboptimal islet cell mass in these patients is associated with reduced morbidity and mortality. The toxic effects of these drugs, however, cause concern about the long-term risks associated with immunosuppression and the need for continuous treatment, particularly in juveniles. Thus, these treatment options are unappealing for an otherwise healthy population of children and young adults with T1DM.

In spite of considerable research to develop pharmacotherapeutic agents that prevent loss of insulin production in T1DM, there is still a great need for such medications. One medication, which received orphan drug status, is an anti-CD3 monoclonal antibody (TRX4, or ChAglyCD3), which was shown to be safe and effective in reducing insulin requirements in new-onset T1DM for at least 18 months in a multicenter trial involving 80 patients (Keymeulen et al., N Engl J Med. 352:2598-2608, 2005). This therapy is associated with a moderate “flu-like” syndrome, requires a six-day intravenous infusion, and has not yet been approved by the U.S. Food and Drug Administration (“FDA”). In the most promising clinical trial in T1DM patients to date, 75% of the patients treated with hOKT3y1(ala-ala), a modified, nonmitogenic, humanized form of the anti-CD3 monoclonal antibody, maintained the same or improved endogenous insulin secretion for one year after an initial 14-day course of therapy (as opposed to 25% in the control group) (Herold et al., New Eng J Med. 346:1692-1698, 2002). But the effect of this therapeutic option appears to wane over time because insulin secretion has declined in this cohort during the second year.

In addition to the clinical trials of the anti-CD3 monoclonal antibody, clinical trials targeting T-cells with cyclosporine (Feutren et al., Lancet 2(8499):119-124, 1986; Dupre and Kolb, Diabetes 37:1574-82, 1988), and azathioprine, alone (Harrison et al., Diabetes 34(12):1306-1308, 1985), or in conjunction with glucocorticoids (Silverstein et al., NEJM 319(10):599-604, 1988), have demonstrated that it is possible to alter the natural course of T1DM in those patients with new-onset of the disease. These clinical trials demonstrated that these agents prolong endogenous insulin secretion in patients, and some participants experienced complete remission. Further and more widespread use of these broad immunosuppressants is limited, however, by the potential complications and toxicities of ongoing therapy, and by the transient nature of their effects, i.e., the therapeutic effects wane despite continued therapy or as drug is withdrawn (Bougneres et al., Diabetes 39(10):1264-1272, 1990; Feldt-Rasmussen et al., Diabet Med. 7:429-433, 1990; Martin et al., Diabetologia. 34:429-434, 1991). Again, such therapies are problematic for children and young adults.

Due to the side-effects, potential toxicity, and limited efficacy of treatments currently available for treating T1DM, particularly in juveniles, it is apparent that there is an urgent need for safer and more effective therapies that preserve endogenous insulin secretion in patients, especially in those newly diagnosed with T1DM. In particular, therapies are needed that provide a means of blocking further autoimmune destruction of the islet cells in patients with new-onset T1DM, thereby promoting retention of endogenous insulin secretion and improving metabolic control. Further, therapies are also needed that could be used to prevent the development of T1DM in patient's at risk for the disease.

### BRIEF SUMMARY OF THE INVENTION

New onset cases of type 1 diabetes mellitus (“T1DM”) generally occur in juveniles, i.e., children and adolescents (approximately 70-85%). The present disclosure relates to the treatment and prevention of T1DM in juveniles (also referred to as juvenile diabetes), for example, by suppressing or inhibiting inflammatory mediators (e.g., inducible nitric oxide synthase (iNOS) and cytokines) by inhibitors of the mevalonate pathway and activators of AMP-activated protein kinase (AMPK). The inhibitors of the mevalonate pathway include but are not limited to inhibitors of synthesis of mevalonate, isoprenoids and isoprenylation of proteins (e.g., small GTPases, Ras/Raf and Rho/Rock-MAPk Kinase cascade). Activators of AMPK include but are not limited to therapeutic agents that enhance the induction or activation of AMPK, which can inhibit signaling cascades for inflammation and immunomodulation. In some embodiments, each therapeutic agent is an inducible nitric oxide synthase (iNOS) and/or proinflammatory cytokine induction suppressor and/or inhibitor, and is administered to the juvenile patient in a biologically effective amount.

In certain embodiments of the present disclosure, therapeutic agents are administered to prevent or decrease the destruction of islet cells, and/or maintain or recover endogenous insulin production in a juvenile patient with T1DM, for example a patient who still endogenously expresses at least some insulin, or who still has at least some functioning islet cells, for example by blocking further autoimmune destruction of islet cells. This prevention or treatment of T1DM is achieved by administering a therapeutic agent to the subject in need, wherein the therapeutic agent (1) inhibits mevalonate synthesis; (2) inhibits the Ras/Raf/MAP kinase or Ras/Rho/MAP kinase pathway, or small GTPase mediated cellular signaling; (3) inhibits the isoprenylation of proteins; (4) inhibits and/or suppresses the induction and/or activation of NF-kβ; (5) inhibits or suppresses the induction of 3-hydroxy-3-methylglutaryl coenzyme A (HMG-CoA) reductase; (6) inhibits or suppresses the induction of mevalonate pyrophosphate decarboxylase (NaPA); (7) inhibits the farnesylation of Ras; (8) inhibits or suppresses the induction of cAMP phosphodiesterase; (9) inhibits or suppresses the induction of farnesyl protein transferase; (10) blocks LPS- and cytokine-induced production of NO by antioxidants; (11) increases or enhances the intracellular levels of cAMP; (12) increases or enhances the activity of AMP protein kinase (AMPK) activity; (13) inhibits dual peroxisome proliferators activated receptor (PPAR) activity; or (14) inhibits the conversion of isopententyl pyrophosphate (IPP) to farnesyl pyrophosphate (FPP). Other embodiments of the present disclosure are directed to treating T1DM is a juvenile patient in need of treatment comprising administering one or more therapeutic agents to the patient in an amount sufficient to regenerate islet cells or islet cell function in the patient.

In certain aspects, the one or more therapeutic agent administered to the juvenile patient are selected from the following: a statin (e.g., lovastatin, atorvastatin (e.g., atorvastatin calcium), simvastatin, pravastatin, cerivastatin, mevastatin, velostatin, fluvastatin, pitavastatin, rosuvastatin, dalvastatin, and fluindostatin), an activator of AMP-activated protein kinase, for example metformin (e.g., metformin hydrochloride), 5-aminoimidazole-4-carboxamide ribonucleoside (AICAR), or a thiazolidinedione (e.g., troglitazon, pioglitazone, or rosiglitazone), an inhibitor of cAMP phophodiesterase (e.g., an inhibitor of phosphodiesterase IV), for example rolipram or PDI-IV, an antioxidant (e.g., N-acetyl cysteine (NAC), S-nitrosoglutathione (GSNO), glutathione, lipoic acid, cafeic acid, or vitamin D), as well as pharmaceutically-acceptable salts, derivatives, analogs, prodrugs, and solvates thereof. In the present disclosure, the therapeutic agents disclosed herein may be administered separately or as a combination of two or more therapeutic agents. When administering two or more of the therapeutic agents, they may be given concomitantly, i.e., so that their biological effects overlap, or concurrently, i.e., within one hour of each other. In addition, at least one other therapeutic agent specific for the treatment of T1DM (e.g., insulin) may be administered with one or more of the therapeutic agents, for example by concomitant administration or coordinated administration.

In certain embodiments, these therapeutic agents allow a juvenile with new-onset T1DM or at risk of developing T1DM to avoid or minimize treatment with insulin injections or insulin pump therapy, thereby reducing chronic complications and premature death, while improving metabolic control and quality of life. In other embodiments, the compounds reduce, delay, or prevent the destruction of islet cells in a patient with T1DM, or a patient at risk for developing T1DM. Since the treatment is for juveniles with T1DM or at risk for T1DM, the safety profile of the therapeutic agent can be relatively benign, particularly when compared to current alternative treatments directed at attenuating autoimmunity in early-onset T1DM.

In certain aspects, the patient population for treatment with the therapeutic agents disclosed herein are juveniles with T1DM who have at least some endogenous insulin production. Even patients with T1DM that potentially have residual insulin production can benefit from treatment with the therapeutic compounds disclosed herein. In addition, patients with T1DM who receive insulin-producing cells through transplantation, for example islet cell transplant, can benefit from treatment as disclosed herein. Currently, it is estimated that between 10,000 and 20,000 juveniles between the ages of 10 and 19 with T1DM have residual insulin secretion. By treating patients who have endogenous insulin production with the therapeutic agents disclosed herein, islet cell function can be preserved in these patients, which can also preserve endogenous insulin production. Loss of islet cells or loss of islet cell function may be determined in a juvenile patient by measuring blood glucose levels, C-peptide levels, and/or insulin levels in the patient. In some embodiments, treatment of patients with new onset T1DM begins within less than two years of diagnosis, or within 12 months, 8 months, 6 months, 4 months, 3 months, 2 months, or 1 month of diagnosis, and or within 12 weeks, 8 weeks, 6 weeks, 5 weeks, 4 weeks, 3 weeks, 2 weeks, or 1 week of diagnosis, or even beginning on the same day as diagnosis. In another embodiment, the therapeutic agents disclosed herein are used at an even earlier stage, for example before the onset of T1DM, to prevent the onset of T1DM in an individual at risk for developing the disease.

An embodiment of the present disclosure is directed to a method of treating type 1 diabetes mellitus in a juvenile patient in need of treatment comprising the steps of:
(1) identifying a juvenile patient diagnosed with type 1 diabetes mellitus with functioning islet cells, and
(2) administering one or more therapeutic agents to the patient in an amount sufficient to maintain or increase the function of the islet cells in the patient,
   wherein the therapeutic agents are selected from the group consisting of an inhibitor of mevalonate synthesis, an inhibitor of 3-hydroxy-3-methylglutaryl coenzyme A (HMG-CoA) reductase, an inducer of AMP protein kinase (AMPK) activity, an inhibitor of dual peroxisome proliferators activated receptor (PPAR) activity, an inhibitor of mevalonic-acid pyrophosphate decarboxylase, an inhibitor of the conversion of isopententyl pyrophosphate (IPP) to farnesyl pyrophosphate (FPP), an inhibitor of the isoprenylation of proteins, an inhibitor of the induction of NF-kβ, an inhibitor of the farnesylation of Ras, an inhibitor of cAMP phosphodiesterase, an antioxidant that blocks LPS- and cytokine-induced production of NO, an enhancer of intracellular levels of cAMP, and any combinations thereof.

Another embodiment of the present disclosure is directed to a method of treating type 1 diabetes mellitus in a juvenile patient in need of treatment comprising the steps of:
(1) identifying a juvenile patient diagnosed with type 1 diabetes mellitus with endogenous insulin secretion, and
(2) administering one or more therapeutic agents to the patient in an amount sufficient to maintain or increase the endogenous insulin secretion in the patient,
   wherein the therapeutic agents are selected from the group consisting of an inhibitor of mevalonate synthesis, an inhibitor of 3-hydroxy-3-methylglutaryl coenzyme A (HMG-CoA) reductase, an inducer of AMP protein kinase (AMPK) activity, an inhibitor of dual peroxisome proliferators activated receptor (PPAR) activity, an inhibitor of mevalonic-acid pyrophosphate decarboxylase, an inhibitor of the conversion of isopententyl pyrophosphate (IPP) to farnesyl pyrophosphate (FPP), an inhibitor of the isoprenylation of proteins, an inhibitor of the induction of NF-kβ, an inhibitor of the farnesylation of Ras, an inhibitor of cAMP phosphodiesterase, an antioxidant that blocks LPS- and cytokine-induced production of NO, an enhancer of intracellular levels of cAMP, and any combinations thereof.

In yet another embodiment of the present disclosure is directed to a method of preventing type 1 diabetes mellitus in a juvenile at risk of developing type 1 diabetes mellitus comprising the steps of:
(1) identifying a juvenile patient at risk for developing type 1 diabetes mellitus, and
(2) administering one or more therapeutic agents to the patient in an amount sufficient to prevent the onset of type 1 diabetes mellitus in the patient,
   wherein the therapeutic agents are selected from the group consisting of an inhibitor of mevalonate synthesis, an inhibitor of 3-hydroxy-3-methylglutaryl coenzyme A (HMG-CoA) reductase, an inducer of AMP protein kinase (AMPK) activity, an inhibitor of dual peroxisome proliferators activated receptor (PPAR) activity, an inhibitor of mevalonic-acid pyrophosphate decarboxylase, an inhibitor of the conversion of isopententyl pyrophosphate (IPP) to farnesyl pyrophosphate (FPP), an inhibitor of the isoprenylation of proteins, an inhibitor of the induction of NF-kβ, an inhibitor of the farnesylation of Ras, an inhibitor of cAMP phosphodiesterase, an antioxidant that blocks LPS- and cytokine-induced production of NO, an enhancer of intracellular levels of cAMP, and any combinations thereof. When the one or more therapeutic agents are administered to the juvenile patient at risk for developing T1DM in an amount sufficient to prevent the onset of T1DM in the patient, the prevention of T1DM may be primary or secondary. Primary prevention preserves islet cell function before the disease process starts, while secondary prevention deters further islet cell destruction or inactivation once it has started and before symptoms of the disease arise.

Another embodiment of the present disclosure is directed to method of treating type 1 diabetes mellitus in a juvenile patient in need of treatment comprising administering one or more therapeutic agents to the patient in an amount sufficient to increase the C-peptide level of the patient after at least six months as compared to the C-peptide level of the patient prior to treatment, wherein the therapeutic agents are selected from the group consisting of an inhibitor of mevalonate synthesis, an inhibitor of 3-hydroxy-3-methylglutaryl coenzyme A (HMG-CoA) reductase, an inducer of AMP protein kinase (AMPK) activity, an inhibitor of dual peroxisome proliferators activated receptor (PPAR) activity, an inhibitor of mevalonic-acid pyrophosphate decarboxylase, an inhibitor of the conversion of isopententyl pyrophosphate (IPP) to farnesyl pyrophosphate (FPP), an inhibitor of the isoprenylation of proteins, an inhibitor of the induction of NF-kβ, an inhibitor of the farnesylation of Ras, an inhibitor of cAMP phosphodiesterase, an antioxidant that blocks LPS- and cytokine-induced production of NO, an enhancer of intracellular levels of cAMP, and any combinations thereof. Methods of determining the juvenile patient's C-peptide levels are well known to those of skill in the art, and may be determined from, for example, samples of the patient's urine or blood. In certain embodiments, the amount of one or more therapeutic agents administered to the juvenile patient is sufficient to increase the C-peptide level of the patient after at least one year, one year and six months, two years, three years, four years, five years, six years, seven years, eight years, nine years, ten year or more as compared to the C-peptide level of the patient prior to treatment.

In certain embodiments of the above methods, the inhibitor of mevalonate synthesis is a competitive inhibitor of 3-hydroxy-3-methylglutaryl coenzyme A (HMG-CoA) reductase such as, for example, a statin. Statins are well known to those of skill in the art, and include, but are not limited to, lovastatin, mevastatin, atorvastatin, fluvastatin, cerivastatin, pitavastatin, pravastatin, rosuvastatin, simvastatin, as well as pharmaceutically-acceptable salts, derivatives, analogs, prodrugs, and solvates thereof. In other embodiments of the above methods, the inducer of AMP protein kinase (AMPK) activity is 5-aminoimidazole-4-carboxamide ribonucleoside (AICAR) or a thiazolidinedione, such as, for example, troglitazone, pioglitazone, or rosiglitazone. In still other embodiments of the above methods, the inhibitor of cAMP phosphodiesterase is rolipram. In other embodiments of the above methods, the antioxidant blocks LPS- and cytokine-induced production of NO, or is selected from the group consisting of N-acetyl cysteine (NAC), S-nitrosoglutathione (GSNO), glutathione, lipoic acid, cafeic acid, and vitamin D. The juvenile that is treated by the above methods may be an adolescent, a pubescent, a pre-pubescent child, or an infant.

Another aspect of the present disclosure is a method of prolonging the honeymoon period of type 1 diabetes mellitus in a juvenile patient in need thereof comprising the steps of:
(1) identifying a juvenile patient in the honeymoon period of type 1 diabetes mellitus, and
(2) administering one or more therapeutic agents to the patient, wherein the therapeutic agents are selected from the group consisting of an inhibitor of mevalonate synthesis, an inhibitor of 3-hydroxy-3-methylglutaryl coenzyme A (HMG-CoA) reductase, an inducer of AMP protein kinase (AMPK) activity, an inhibitor of dual peroxisome proliferators activated receptor (PPAR) activity, an inhibitor of mevalonic-acid pyrophosphate decarboxylase, an inhibitor of the conversion of isopententyl pyrophosphate (IPP) to farnesyl pyrophosphate (FPP), an inhibitor of the isoprenylation of proteins, an inhibitor of the induction of NF-kβ, an inhibitor of the farnesylation of Ras, an inhibitor of cAMP phosphodiesterase, an antioxidant that blocks LPS- and cytokine-induced production of NO, an enhancer of intracellular levels of cAMP, and any combinations thereof,
   wherein the administration of the one or more therapeutic agents results in a prolonged honeymoon period in the juvenile patient. In certain aspects, the juvenile patient in the honeymoon period requires less than 0.5 U/kg/day of insulin and/or has a hemoglobin A1c level equal to or less than 6%.

Thr honeymoon period for the juvenile patient can be prolonged by administering an inhibitor of mevalonate synthesis, for example a competitive inhibitor of 3-hydroxy-3-methylglutaryl coenzyme A (HMG-CoA) reductase such as, for example, a statin, including, but are not limited to, lovastatin, mevastatin, atorvastatin, fluvastatin, cerivastatin, pitavastatin, pravastatin, rosuvastatin, simvastatin, as well as pharmaceutically-acceptable salts, derivatives, analogs, prodrugs, and solvates thereof, and combinations thereof. In other embodiments of this method, the inducer of AMP protein kinase (AMPK) activity is 5-aminoimidazole-4-carboxamide ribonucleoside (AICAR) or a thiazolidinedione, such as, for example, troglitazone, pioglitazone, or rosiglitazone. In still other embodiments this method, the inhibitor of cAMP phosphodiesterase is rolipram. In other embodiments of this method, the antioxidant blocks LPS- and cytokine-induced production of NO, or is selected from the group consisting of N-acetyl cysteine (NAC), S-nitrosoglutathione (GSNO), glutathione, lipoic acid, cafeic acid, and vitamin D. The juvenile that is treated by the above methods may be an adolescent, a pubescent, a pre-pubescent child, or an infant.

Another embodiment of the present disclosure is directed to a method of treating type 1 diabetes mellitus in a juvenile patient in need of treatment comprising administering one or more statins, or pharmaceutically-acceptable salts, derivatives, analogs, prodrugs, and solvates thereof, to the patient in an amount sufficient to increase the C-peptide level of the patient after at least six months as compared to the C-peptide level of the patient prior to treatment. The statin may be selected from the group consisting of lovastatin, mevastatin, atorvastatin, fluvastatin, cerivastatin, pitavastatin, pravastatin, rosuvastatin, simvastatin, as well as pharmaceutically-acceptable salts, derivatives, analogs, prodrugs, and solvates thereof, and combinations thereof. The juvenile that is treated by this method may be an adolescent, a pubescent, a pre-pubescent child, or an infant. In certain aspects, the amount of one or more statins, or pharmaceutically-acceptable salts, derivatives, analogs, prodrugs, and solvates thereof, administered to the juvenile patient is sufficient to increase the C-peptide level of the patient after at least one year, one year and six months, two years, three years, four years, five years, six years, seven years, eight years, nine years, or ten year or more as compared to the C-peptide level of the patient prior to treatment. In certain aspects, the ratio of the C-peptide level of the patient after treatment compared to the C-peptide level of the patient prior to treatment is at least about or up to about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.5, 5.0, 5.5, 6.0, 6.5, 7.0, 7.5, 8.0, 8.5, 9.0, 9.5, or 10.0, to 1.0. In other embodiments, the C-peptide level of the patient after treatment compared to the C-peptide level of the patient prior to treatment increases at least about 1.1-fold, 1.2-fold, 1.3-fold, 1.4-fold, 1.5-fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.9-fold, 3.0-fold, 3.1-fold, 3.2-fold, 3.3-fold, 3.4-fold, 3.5-fold, 3.6-fold, 3.7-fold, 3.8-fold, 3.9-fold, 4.0-fold, 4.5-fold, 5.0-fold, 5.5-fold, 6.0-fold, 6.5-fold, 7.0-fold, 7.5-fold, 8.0-fold, 8.5-fold, 9.0-fold, 9.5-fold, or 10.0-fold.

In other embodiments, the present disclosure is directed to a pharmaceutical composition such as, for example, a single dosage form, i.e., in a unit dosage form, useful in preventing or treating T1DM in a juvenile patient, which comprises one or more therapeutic agents described herein. In some embodiments, the compositions are adapted for oral, intranasal, intravenous, parenteral, pulmonary, transdermal, buccal, or sublingual administration. In certain embodiments, the unit dosage form may be either a tablet, capsule, suppository, parenteral, or other. Other excipients may also be present in the dosage form, such as pregelatinized maze starch, polyvinyl-pyrrolidone or hydroxypropyl methylcellulose; fillers (e.g., lactose, microcrystalline cellulose or calcium phosphate); disintegrants (e.g., potato starch, croscarmellose sodium, or sodium starch glycollate); wetting agents (e.g., sodium lauryl sulphate), or other agents for tableting. In other embodiments, the compositions comprising therapeutic agents, individually or in combination, are employed in admixture with conventional excipients, i.e., pharmaceutically acceptable organic or inorganic carrier substances suitable for parenteral, enteral (e.g., oral or intranasal) or topical application which do not deleteriously react with the active compositions. The pharmaceutical preparations can be sterilized and, if desired, mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the therapeutic agents. For parenteral application, particularly suitable are injectable, sterile solutions, such as, for example, oily or aqueous solutions, as well as suspensions, emulsions, or implants, including suppositories. Ampules, vials, and injector cartridges are convenient unit dosages.

Sustained or directed release compositions can also be formulated, e.g., liposomes or compositions in which the active component is protected with differentially degradable coatings, e.g., by microencapsulation, multiple coatings, etc. It is also possible to freeze-dry the new compositions and use the lyophilizates obtained, for example, for the preparation of products for injection. The actual amounts of active compositions in a specific case will vary according to the specific compositions being utilized, the particular compositions formulated, the mode of application, the particular route of administration, the age of the juvenile patient, and the status of the juvenile's disease. Dosages for a given juvenile can be determined using conventional considerations, e.g., by means of an appropriate, conventional pharmacological protocol.

Another embodiment of the present disclosure is a therapeutic package for dispensing to, or for use in dispensing to, a juvenile patient with T1DM, which comprises: (a) one or more unit dosage forms, each unit dosage form comprising one or more therapeutic agent as disclosed herein, wherein each therapeutic agent may be in a separate unit dosage form, and/or a combination of therapeutic agents may be in a single unit dosage form; and (b) a finished pharmaceutical container therefore, said container containing the unit dosage form or unit dosage forms, and further comprising labeling directing the use of said package in the treatment of T1DM.

It is contemplated that any embodiment discussed in this specification can be implemented with respect to any compound, method, or composition of the invention, and vice versa.

The terms “about” or “approximately” are defined as being close to as understood by one of ordinary skill in the art, and in one non-limiting embodiment the terms are defined to be within 10%, within 5%, within 1%, or within 0.5%.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5%.

The terms “inhibiting,” “reducing,” or “prevention,” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an” when used in conjunction with the term “comprising” in the claims and/or the specification may mean “one,” but it is also consistent with the meaning of “one or more,” “at least one,” and “one or more than one.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

### BRIEF DESCRIPTION OF THE SEVERAL VIEWS OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1. NOD mice treated with saline, 5 mg/kg Atorvastatin calcium, or 10 mg/kg Atorvastatin calcium, surviving (disease-free) after 104 days of treatment.

FIG. 2. NOD mice treated with saline, 5 mg/kg Atorvastatin calcium, or 10 mg/kg Atorvastatin calcium, surviving (disease-free) after 109 days of treatment.

FIG. 3. NOD mice with diabetes treated with saline, 5 mg/kg Atorvastatin calcium, or 10 mg/kg Atorvastatin calcium, after 83 days of treatment.

FIG. 4. NOD mice without diabetes treated with saline, 5 mg/kg Atorvastatin calcium, or 10 mg/kg Atorvastatin calcium, after 83 days of treatment.

FIG. 5. NOD mice without diabetes treated with saline, 5 mg/kg Atorvastatin calcium, or 10 mg/kg Atorvastatin calcium, after 103 days of treatment.

FIG. 6. NOD mice without diabetes treated with saline, 5 mg/kg Atorvastatin calcium, 10 mg/kg Atorvastatin calcium, or 10 mg/kg AICAR; data shows up to a 75% reduction in diabetes in NOD mice.

FIG. 7. NOD mice treated with saline, 5 mg/kg Atorvastatin calcium, 10 mg/kg Atorvastatin calcium, or AICAR surviving (disease-free) after 104 days of treatment.

FIG. 8. Blood glucose (mg/dl) levels were measured in NOD mice treated as follows: (1) received vehicle only by oral lavage daily (Saline); (2) received atorvastatin calcium daily at an oral dose of 5 mg/kg body weight (Lipitor 5); (3) received atorvastatin calcium daily at an oral dose of 10 mg/kg body weight (Lipitor 10); (4) received AICAR daily at an oral dose of 0.5 mg/gm body weight (Aicar); and (5) received a combination of atorvastatin calcium daily at an oral dose of 10 mg/kg body weight and AICAR daily at an oral dose of 0.5 mg/gm body weight (L+A).

FIG. 9. Effects of Atorvastatin calcium treatment, 5 mg/kg body weight or 10 mg/kg body weight, and 30 mg/kg body weight AICAR treatment on induction of pro-inflammatory cytokines and iNOS in NOD mice.

FIG. 10. Lower levels of islet cell inflammation found in the NOD mice protected with simvastatin.

FIG. 11. Simvastatin treatment protected insulin producing islet cells and reduced inflammatory cells around and in the islet cells in the pancreas of NOD mice.

FIG. 12. Simvastatin treatment showed a significant increase in the number of insulin producing islet cells in the pancreas of NOD mice as compared to those mice treated with saline.

FIG. 13. Simvastatin treatment showed an increase in insulin message level in the pancreas of NOD mice.

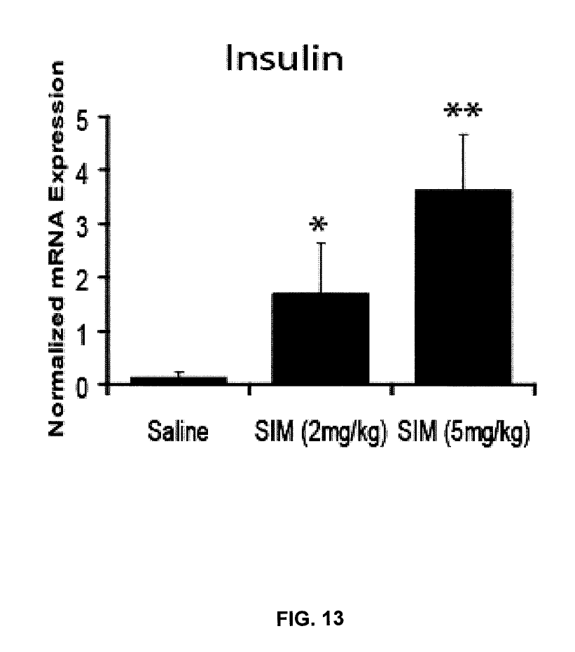

FIG. 14. Graph showing the actual versus expected ratio of urinary C-peptide in an 11-year old human patient with positive insulin antibodies initially treated for two weeks after diagnosis with 10 mg Atorvastatin calcium, and subsequently treated with 20 mg Atorvastatin calcium.

FIG. 15. Graph showing the actual versus expected ratio of urinary C-peptide in a 17-year old human patient with positive insulin antibodies treated with 20 mg Atorvastatin calcium.

### DETAILED DESCRIPTION OF THE INVENTION

The present disclosure is generally directed to methods of treating type 1 diabetes mellitus (T1DM) in juveniles, or preventing or delaying the onset of T1DM in juveniles at risk for the disease. The present disclosure provides methods of preventing or treating T1DM in juveniles by administering one or more compounds that block the loss or further loss of islet cells, or aid in the retention or recovery of endogenous insulin secretion. As used herein, the terms “therapeutically,” “to treat,” “treatment,” or “therapy” refer to both therapeutic treatments and prophylactic or preventative measures. In the present disclosure, the phrase “islet cells” may be used interchangeably with the phrase “beta cells,” since beta cells are a subset of cells found within the islet cells. In certain embodiments, this prevention or treatment of T1DM can be achieved by administering one or more therapeutic agents to the subject in need thereof, wherein the therapeutic agent is a competitive inhibitor of mevalonate synthesis, a competitive inhibitor or suppressor of the induction of 3-hydroxy-3-methylglutaryl coenzyme A (HMG-CoA) reductase, an inducer of AMP protein kinase (AMPK) activity, an inhibitor of dual peroxisome proliferators activated receptor (PPAR) activity, an inhibitor or suppressor of mevalonic-acid pyrophosphate decarboxylase (NaPA), an inhibitor of the conversion of isopententyl pyrophosphate (IPP) to farnesyl pyrophosphate (FPP), an inhibitor or suppressor of the Ras/Raf/MAP kinase or Ras/Rho/MAP kinase pathway, or small GTPase mediated cellular signaling, an inhibitor or suppressor of the isoprenylation of proteins, an inhibitor or suppressor of the induction and/or activation of NF-kβ, an inhibitor or suppressor of the farnesylation of Ras, an inhibitor or suppressor of the induction of cAMP phosphodiesterase, an inhibitor or suppressor of the induction of farnesyl protein transferase, an antioxidant that blocks LPS- and cytokine-induced production of NO, an enhancer of intracellular levels of cAMP.

In certain embodiments, pharmaceutically-acceptable salts, derivatives, analogs, prodrugs, or solvates of the therapeutic compounds disclosed herein can be used in the methods of the present disclosure. Treating juvenile patients suffering from T1DM with the therapeutic agents disclosed herein will improve diabetes control, lessen the likelihood of complications, and improve the quality of life for patients with T1DM. In the methods of the present disclosure, the compounds may be administered separately or in combination with one or more other compounds disclosed herein. The compounds disclosed herein may also be administered in combination with insulin. In addition, the compounds may be given concomitantly, i.e., so that their biological effects overlap, or concurrently, i.e., within one hour of each other.

There is evidence that T1DM is a Th1-cell mediated autoimmune disease, where infiltrating vascular immune cells and the resulting local induction of inflammatory mediators are thought to play a role in islet cell pathology (Wen et al., J Exp Med. 191:97-104, 2000; Keller, R J, J Autoimmun. 3:321-327, 1990; Nakayama et al., Nature 435:220-223, 2005; Ozawa et al., J Autoimmun. 9:517-524, 1996). In NOD mice, lymphocytic infiltration surrounding the islets is the initial pathological finding occurring at 5-8 weeks. These infiltrates contain many types of inflammatory cells including antigen presenting cells (including macrophages), T-helper cells, cytotoxic T-cells, B-lymphocytes and natural killer cells (Yang and Santamaria, Clin Sci (Lond) 110:627-639, 2006). In NOD mice, the role of Th-1 cells in inducing inflammatory mediators, particularly TNF-α, IL-β and IFN-γ, is well documented in islet cell death and resulting insulitis (Wen et al., J Exp Med. 191:97-104, 2000; Yang and Santamaria, Clin Sci (Lond) 110:627-639, 2006; Muir et al., J Clin Invest. 95:628-634, 1995; Shimada et al., Diabetes 45:65-169, 1996).

A number of recent reports have also supported the role of Th-1 cells and their inflammatory mediators in T1DM in humans (Almawi et al., J Clin Endocrinol Metab. 84:1497-1502, 1999; Azar et al., Clin Diagn Lab Immunol. 6:306-310, 1999). Our understanding of this process has been evolving rapidly and along with it, strategies of immune therapy. One particular strategy relevant to the proposed studies is the shifting of T-cell phenotype from Th-1 to Th-2 (Falcone and Bloom, J Exp Med. 185:901-907, 1997; Stanislaus et al., Neurosci Lett. 333:167-170, 2002), and attenuation of inflammatory mediated cellular insult/loss with immune response modifying agents such as statins and activators of AMP-activated Kinase (AICAR) (Stanislaus et al., Neurosci Lett. 269:71-74. 1999; Stanislaus et al., Neurosci Lett. 333:167-170, 2002; Stanislaus et al., J Neurosci Res. 66:155-162, 2001; Giri et al., J Neurosci. 24:479-487, 2004). Statins used in the active phase of experimental autoimmune encephalomyelitis in an animal model for multiple sclerosis, which is a Th1 cell-mediated disease, have shown benefit (Youssef et al., Nature 420:78-84, 2002; Nath et al., J Immunol. 172:1273-1286, 2004).

Without being bound by any particular theory, it is thought that shifting islet-reactive T-cells from a Th1 to a Th2 phenotype will prevent the onset or delay the progress of T1DM. In certain embodiments, it is thought that the therapeutic agents administered according to the methods of the present disclosure decrease islet cell death by regulating upstream mediators of T-cell phenotype. It appears that by inhibiting or inducing these regulators appropriately, autoreactive T-cells can be shifted from a Th1 to a Th2 phenotype. Shifting the phenotype of the T-cells appears to result in decreased destruction of islet cells in subjects treated with a therapeutically effective amount of one or more upstream mediators of the T-cell phenotype. As used herein, a “therapeutically effective amount” of cells or tissues is an amount sufficient to arrest or ameliorate the physiological effects in a subject caused by the loss, damage, malfunction, or degeneration of particular cell-types or tissue-types, including, but not limited to, islet cells.

Certain embodiments of the present disclosure are directed to methods of treating type 1 diabetes mellitus (T1DM) in a juvenile patient in need of such treatment, or preventing or delaying the onset of T1DM in a juvenile patient at risk for the disease, comprising administering to the patient a biologically effective amount of an HMG-CoA reductase or a pharmaceutically acceptable salt thereof. Therapeutic compounds that are HMG-CoA reductase inhibitors include, but are not limited to, statins. Statins were originally developed for the treatment of hypercholesterolemia, and are competitive inhibitors of HMG-CoA reductase, the enzyme that catalyzes the conversion of HMG-CoA to mevalonate. Because mevalonate is required for cholesterol synthesis, HMG-CoA reductase is an early and rate-limiting step in the biosynthesis of cholesterol. HMG-CoA reductase inhibitors are the most commonly used agents in the treatment of hypercholesterolemia. Examples of statins include, but are not limited to, vastatins such as simvastatin (e.g., Zocor®, Lipex), disclosed in U.S. Pat. No. 4,444,784; pravastatin (e.g., Pravachol®, Selektine, Lipostat), disclosed in U.S. Pat. No. 4,346,227; cerivastatin (e.g., Baycol®, Lipobay), disclosed in U.S. Pat. No. 5,502,199; mevastatin, disclosed in U.S. Pat. No. 3,983,140; velostatin, disclosed in U.S. Pat. Nos. 4,448,784 and 4,450,171; fluvastatin (e.g., Lescol®), disclosed in U.S. Pat. No. 4,739,073; compactin, disclosed in U.S. Pat. No. 4,804,770; lovastatin (e.g., Mevacor®, Altocor™), disclosed in U.S. Pat. No. 4,231,938; pitavastatin (e.g., Livalo®, Pitava); rosuvastatin (e.g., Crestor®); dalvastatin, disclosed in European Patent Application Publication No. 738510 A2; fluindostatin, disclosed in European Patent Application Publication No. 363934 A1; atorvastatin, disclosed in U.S. Pat. No. 4,681,893; atorvastatin calcium (e.g., Lipitor® or Torvast), disclosed in U.S. Pat. No. 5,273,995; and dihydrocompactin, disclosed in U.S. Pat. No. 4,450,171. Based on the anti-inflammatory properties of statins, it is thought that statin treatment will attenuate/inhibit disease processes in juveniles suffering from T1DM, or at risk for T1DM, for example by inducing a shift of Th1 to Th2 phenotype, protecting islet cell functions, protecting against the loss of islet cells, maintaining or increasing endogenous insulin secretion, inhibiting induction of proinflammatory cytokines and inducible nitric oxide synthase, inducing PPARy, and protecting cells against inflammatory cellular insult. In certain embodiments, these statins, as well as pharmaceutically-acceptable salts, derivatives, analogs, prodrugs, and solvates thereof, can be used in the methods of the present disclosure.

Another class of therapeutic agents that inhibit HMG-CoA reductase are the AMPK inducers, which include compounds such as 5-Aminoimidazole-4-carboxamide-1-β-D-ribofuranoside 5′-monophosphate (AICAR), ZMP (5-Aminoimidazole-4-carboxamide-1-β-D-ribofuranosyl 5′-monophosphate), biguanides, including but not limited to phenformin and metformin (e.g., metformin hydrochloride), or thiazolidinediones (e.g., troglitazon, pioglitazone, or rosiglitazone), as well as pharmaceutically acceptable salts, derivatives, analogs, prodrugs, and solvates thereof. This class of compounds inhibits HMG-CoA reductase by increasing the activity of AMPK, which down-regulates HMG-CoA reductase activity by phosphorylation. AMPK has been extensively studied for its activity in carbohydrate and lipid metabolism. It also plays a role in inflammatory disease processes by protecting the endothelial function and inhibition of induction of inflammatory cytokines (Giri et al., J Neurosci. 24:479-487, 2004). One report has shown that modulation of the Th1/Th2 axis in a remitting-relapsing EAE model of MS by AICAR may be mediated via AMPK activation (Nath et al., J Immunol 175:566-574, 2005). Activation of AMPK with AICAR attenuated the inflammatory disease of EAE (Prasad et al., J Neurosci Res. 84:614-25, 2006) and provided protection in organ preservation and kidney transplantation (Lin et al., Transplantation 78:654-659, 2004).

Without being bound by any particular theory, interruption of the mevalonate pathway is believed also to result in anti-inflammatory activity due to the reduction of intermediary metabolites (such as isoprenoids), which are produced by the mevalonate pathway, rather than by the depletion of cholesterol end products of the melavonate pathway. HMG-CoA reductase converts HMG-CoA to mevalonate. Mevalonate in turn is converted to mevalonate pyrophosphate, which is then converted by mevalonic-acid pyrophosphate decarboxylase to IPP. IPP and its isomer dimethylallylpyrophosphate are precursors of the isoprenyl groups FPP and geranylgeranylpyrophosphate (GGPP). Farnesylation or geranylgeranylation of various small G-proteins (collectively referred to as isoprenylation) is a necessary step in activating these proteins which are important regulatory GTPases. G-proteins are sub-divided into at least six families or sub-families: (1) Ras, including Ras, Rap, Rad, Ral, Rin and Rit, (2) Rho, including Rho, Rac, Cdc42, and Rnd, (3) Rab, (4) Sar1/ADP ribolsylation factor, including Arf, Arl, Ard and Srl, (5) Ran, and (6) Rad. Prenylation is required to activate many of these G-proteins and is a necessary step for translocating small G-proteins such as the Ras and Rho G-proteins. Ras and Rho are the central molecules upstream of the Ras/Raf/MAP kinase cascade.

A downstream effect of the activation of G-proteins is the induction of proinflammatory cytokines (IL-1β, TNFα and IFN-γ) and of inducible nitric oxide synthase (iNOS) in cells such as macrophages, T-cells, astrocytes and microglia. Induction of proinflammatory cytokines and/or the nitric oxide (NO) produced by iNOS are believed to repress the shift of Th1 to Th2 cells. Without being bound by any particular theory, it is believed that promoting the shift of Th1 to Th2 cells will prevent or decrease destruction of islet cells in juvenile patients with T1DM. Thus, certain aspects of the present disclosure involve the repression or induction of steps in the pathway described above that result in promotion of the shift of Th1 cells to Th2 cells.

Additionally, as a result of inhibiting the mevalonate pathway, statins also down-regulate the activity of dual peroxisome proliferators activated receptors (PPAR), including PPAR-γ. Inhibition of PPAR either by inhibiting the mevalonate pathway or by the use of PPAR agonists is believed to inhibit iNOS protein activity and thus promote the shift of Th1 to Th2 cells through this pathway. PPAR agonists include the thiazolidinedione class of drugs, also called the glitazones, which include but are not limited to troglitazone, pioglitazone, ciglitazone, englitazone and rosiglitazone. These therapeutic agents, as well as pharmaceutically-acceptable salts, derivatives, analogs, prodrugs, and solvates thereof, may be used in certain embodiments of the present disclosure.

Therefore, certain aspects of the present disclosure involve the administration of a therapeutically-effective amount of one or more therapeutic agents that (1) inhibits PPAR either through blocking the mevalonate pathway or by administration of PPAR agonists; (2) induce AMPK to block the mevalonate pathway; (3) inhibit mevalonic-acid pyrophosphate decarboxylase, for example by administration of sodium phenylacetate or sodium phenylbutyrate; (4) inhibit FPP synthesis by inhibiting the conversion of IPP to FPP, for example through the use of FPT inhibitor II; (5) inhibit iNOS and/or inflammatory cytokine activity; or (6) inhibit cAMP phophodiesterase (e.g., an inhibitor of phosphodiesterase IV), for example by the administration of rolipram or PDI-IV. The therapeutic agents disclosed herein are administered to prevent or decrease the destruction of islet cells and/or maintain or recover endogenous insulin production in a juvenile patient with T1DM, for example, a patient who still endogenously expresses at least some insulin, or who still has at least some functioning islet cells.

The present disclosure describes methods of administering one or more of the therapeutic agents described above in a therapeutically-effective amount to a juvenile subject at risk for or suffering from T1DM. A juvenile patient at risk for or suffering from T1DM may be diagnosed based on the clinical presentation of hyperglycemia (e.g., a fasting glucose level of greater than 123 mg/dl), mild ketosis or diabetic ketoacidosis, and/or the presence of insulin or islet cell autoantibodies in the patient. In certain embodiments the subject is a juvenile that is in either the “honeymoon period” of T1DM, or who has been identified as being at risk for T1DM. As used herein, a “subject” or a “patient,” which are terms that may be used interchangeably, may be a juvenile animal, such as a mammal, including, but not limited to, humans, pigs, cats, dogs, rodents, sheep, goats and cows. In some aspects, the subject or patient is juvenile human. As used herein, the term “juvenile” includes infants, pre-pubescent children, pubescent children, and adolescents. An infant is a child under the age of one year. With regard to pre-pubescent, pubescent and adolescent juveniles, the stage-of-life of a child over the age of one year can be assessed using the Tanner-stage scale. Tanner-stage criteria are well-known to those of skill in the art, and are specific to the gender of the patient. Tanner-stage 1 defines the physical characteristics of a pre-pubescent male or female child. Tanner-stages 2-4 define the physical characteristics of a pubescent male or female child. Tanner-stage 5 defines the physical characteristics of an adolescent. Adolescents therefore are post-pubertal individuals that have not yet reached adulthood. In certain embodiments, treatments described herein are begun prior to adulthood in patients with early-onset T1DM, or who are at risk for T1DM. It may be highly desirable to continue treatment as disclosed herein for a patient with T1DM into adulthood, or to treat patients with adult-onset T1DM.

As used herein, the “honeymoon period” of T1DM refers to a period immediately after the onset of the disease, i.e., immediately after some portion of a patient's pancreatic islet cells have undergone injury or destruction. The honeymoon period is typically identified after symptoms of the disease are first noted, but also may include early stages of the disease in which the injury or destruction of islet cells has yet to produce noticeable symptoms in the patient or the need for insulin therapy. During the honeymoon period, patients retain the ability to secrete significant amounts of insulin, possibly because the undamaged islet cells in the individual are induced to work harder. This may result in the individual not needing administration of insulin or needing very low levels of insulin at this time to control the disease. Therefore, the honeymoon period alternatively may be referred to as “clinical remission” or “partial remission” of the disease. The honeymoon period may last for days, weeks, or even years, although in the majority of individuals it does not last longer than one year. In those diagnosed with T1DM as an infant or pre-pubescent, the honeymoon period is more likely to be shorter or even absent than for older individuals. The honeymoon period is considered to have ended when islet cell function has reached trace or undetectable levels and the patient is therefore completely reliant on the administration of exogenous insulin. In certain aspects, the honeymoon period is defined as a period with insulin requirements of less than 0.5 U/kg/day and hemoglobin A1c (HbA1c) level of less or equal to 6%.

In some embodiments, the methods of the present disclosure will extend the honeymoon period of T1DM, as evidenced by a statistically significant increase in the length of the honeymoon period in an individual patient or in a group of patients treated with one or more of the compounds disclosed herein versus a control group of untreated or conventionally-treated patients having similar characteristics. The methods of the present disclosure will extend the length of the honeymoon period in T1DM patients for a period of at least about 6 months, 12 months, or 18 months, or for at least about 2 years, 3 years, 4 years, 5 years, 6 years, 7 years, 8 years, 9 years, 10 years, or longer. In some embodiments the administration of the identified therapeutic agents not only extends the honeymoon period of T1DM, but also results in preservation of islet cell function and/or endogenous insulin secretion in the patient for a statistically-significant period of time.

A marker for diagnosing T1DM is C-peptide. C-peptide is made when proinsulin is split into insulin and C-peptide, which occurs when proinsulin is released from the pancreas into the blood in response to increased serum glucose. Since C-peptide is excreted in equimolar ratios to insulin, it can distinguish between a diagnosis of T1DM and Type 2 diabetes. In T1DM, C-peptide levels are measured instead of the insulin levels because insulin concentration in the portal vein ranges from two to ten times higher than in the peripheral circulation. For example, he amount of insulin in the plasma extracted by the liver varies with nutritional state. Since the pancreas of a patient with T1DM is unable to produce insulin, or produces reduced amounts of insulin, this patient will usually have a decreased level of C-peptide, or an absence of C-peptide. In contrast, C-peptide levels in patients with type 2 diabetes is normal or higher than normal. Measuring C-peptide in T1DM patients injecting insulin can help determine how much endogenous insulin these patients are still producing.

At this time, only C-peptide has been shown to be elevated in prediabetic patients, and it has been suggested that this may be a marker for the risk of progression to clinical disease (Chase et al., Diabetes 53(10):2569-73, 2004). Interleukin-6 and TNF-alpha levels are also elevated in newly diagnosed patients with T1DM, but the relevance of these findings to the etiology of the disease remains unclear (Davi et al., Circulation 107:3199-3203, 2003; Scholin et al., Diabetes/Metabolism Research and Reviews 20:205-210; 2004; Erbagci et al., Clinical Biochemistry. 24:645650; 2001). Determining whether a juvenile patient is still in the honeymoon phase or has advanced to the point where the patient has little or no islet cell function left may be accomplished using a number of protocols well known to those of skill in the art, including, for example, C-peptide response to fasting, intravenous glucagon, and mixed-meal tolerance test (MMTT) (e.g., 2-hour MMTT or 4-hour MMTT). The MMTT functions as a stimulated C-peptide level test. Serial assessment of endogenous insulin secretion may be measured by the C-peptide area under the curve (AUC) in response to a 2-hour MMTT or 4-hour MMTT.

The efficiency of a particular therapeutic agent disclosed herein at preserving islet cell function may be assessed by a comparison of base-line AUC with the AUC after 12-months of treatment. The efficacy may also be assessed by comparing the actual ratio of C-peptide compared to the expected ratio of C-peptide after 12-months. C-peptide levels that indicate that a patient has impaired islet cell function, or that a patient has no significant islet cell function remaining, and thus has exited the honeymoon period of the disease into the end-stage of T1DM, are well known to those of skill in the art. See, e.g., Kusayanagi, T., Nippon Ika Daigaku Zasshi 56(2):103-22, 1989; Aurbach-Klipper et al., Diabetologia 24:88-90, 1983; Pasquali et al., Diabete Metab. 13:44-51, 1987; and De Beaufort et al., Diabet. Med. 5:441-43, 1988; each of which is incorporated herein in its entirety. Likewise, expected reduction in C-peptide levels after the onset of T1DM are well known to those of skill in the art. C-peptide levels may be measured either in collected samples of plasma or urine. A good correlation between the levels of plasma C-peptide and urinary C-peptide values as related to creatinine has been found. Pasquali et al., Diabete Metab. 13:44-51, 1987. Therefore, since it is simpler and less traumatic to obtain urine samples from children than blood samples, it may be useful to evaluate urinary C-peptide values in the majority of juvenile patients.

The following is a general description of the protocol for the glucagon test, which can be modified as appropriate by one of skill in the art. Subjects should be kept supine for the duration of the test. After basal blood samples for glucose and C-peptide are taken, for example using an indwelling venous cannula kept patent with normal saline, glucagon (e.g., 1 mg diluted in 1 ml of normal saline) is injected intravenously into the patient over 1 minute. A second glucagon bolus may be injected 30 minutes after the first stimulus. Blood samples are then taken at intervals of 2, 5, 10, 20, and 30 minutes following the two stimuli. Vannini et al., Int. J. Obesity 6:327-34, 1982, which is incorporated herein by reference. The aliquots of sera may be stored at −20° C. until analysis.

The following is a general description of the protocol for the MMTT, which can be modified as appropriate by one of skill in the art. When conducing a MMTT of a patient, the patient prepares for the procedure by fasting overnight. Immediately prior to the test, the patient should be instructed to: (1) fast from all food and drinks (including coffee, teas, and diet drinks), except for water, for ideally 10 hours but no less than 8 hours prior to their scheduled appointment time; (2) abstain from tobacco products and vigorous exercise for 10 hours prior to the scheduled appointment time; (3) insulin-dependent patients should be maintained on their current insulin regimen until the evening before the day of study when they will receive only regular insulin (or short acting insulin analogue) before supper and again with a snack before bedtime; in addition, the evening long- or intermediate-acting insulin and the morning insulin should be withheld; (4) if the patient uses an insulin pump, the patient will maintain his or her usual basal insulin rates until 3 hours prior to the test or 5 hours if using buffered regular insulin; at this point blood sugar should be checked, and the pump suspended (after a modest bolus for correction of hyperglycemia).

For safety reasons, it is recommended that the patient's blood sugar level be within the range of 60-250 mg/dL prior to beginning the MMTT. If the patient experiences low blood sugar (<60 mg/di or <80 mg/di with symptoms) on the morning of the stimulated C-peptide test, the patient should be given a fast-acting carbohydrate (e.g., 3-4 oz. of fruit juice), and the test should be rescheduled. If the patient experiences high blood sugar on the morning of the stimulated C-peptide test, the patient may take a small dose of rapid-acting insulin according to his or her usual routine. The test can be performed 3 hours after the rapid insulin has been taken, provided the blood glucose is within the range of 60-250 mg/dL, if any diabetes medications were taken the morning of the test. Rapid-acting insulin (Humalog or Novolog) or an insulin bolus by pump may be given the morning of the test, provided at least 3 hours elapse between the administration of insulin and the start of the test.

The C-peptide test itself may be performed as follows: (1) the patient's blood glucose and body weight are recorded; (2) a liquid test meal consisting of 6 ml/kg body weight of Boost® High Protein (Mead Johnson) or other equivalent liquid meal containing a standard amount of fat, protein, and carbohydrate are administered up to a maximum of 360 ml; Boost® High Protein is available in 237 ml cans, and one can contain 240 calories and the composition is 24% protein, 55% carbohydrate, and 21% fat; (3) blood glucose and C-peptide samples are obtained from the patient at ten specific time intervals (at baseline and after 15, 30, 60, 90, 120, 150, 180, 210, and 240 minutes).

Prior to obtaining specimens, the patient should rest quietly in a supine or seated position. An intravenous catheter (e.g., 20 to 22 gauge) is inserted into a large antecubital vein. Local anesthesia may be used, but is not required. A baseline sample should be obtained at least 10 minutes after establishing venous access when the patient is calm and relaxed. This is considered the “0” minute sample. The patient is then asked to drink the liquid test meal. The drink should be completely consumed as soon as possible, in no more than 5 minutes. The time clock is started once the drink is completed. Post-meal samples are obtained at specified times after the clock is started, and the actual time each of these samples is drawn is recorded. The test is complete after the 240-minute sample is drawn. The intravenous catheter is removed and pressure is applied until bleeding stops. The patient may then be assisted in checking his or her blood glucose, and administering an appropriate insulin dose. The patient may then eat a breakfast meal.

Blood samples are kept on ice for 30 minutes or less before processing. Samples are centrifuged, the supernatant removed, and Trasylol, 500 klU per 1 ml plasma (125 μl Aprotinin/ml) is added. Glucose is measured immediately on individual samples. Aliquots for C-peptide analysis can be stored at −70° C. for later analysis. C-peptide can be measured using a commercially available RIA (Limo Research Inc.).

Success of treatment by a particular therapeutic agent may be defined by the number of subjects who preserve islet cell function as measured at baseline (prior to treatment) and 12-months. Preservation of islet cell function is indicated by a less than 7.5% decrease from baseline in the total area under the curve for a 4-hour MMTT of C-peptide levels (7.5% being half of the interassay coefficient of variation for the C-peptide level assay). The 4-hour MMTT is sometimes preferred over the 2-hour MMTT in light of the observation many subjects with impaired islet cell function do not reach a peak in C-peptide value during the first 2 hours (Greenbaum and Harrison, Diabetes, 52:1059-1065, 2003, incorporated herein by reference). While published studies have indicated that approximately 17% of individuals treated conventionally for T1DM will preserve islet cell function after twelve months, the remaining 83% will see a significant decrease in islet cell function (Herold et al., New Eng J Med. 346:1692-1698, 2002). Administration of one or more of the compounds as disclosed herein, for example a statin, will increase the percentage of individuals retaining islet cell function and/or endogenous insulin secretion after 12-months versus a control group treated conventionally. For example, these patients will retain at least about 99%, 95%, 90%, 80%, 70%, 60%, 50%, 40%, 30%, 20% or 10% of islet cell function and/or endogenous insulin secretion after 12-months as compared to a control group. Similarly, embodiments of the present disclosure will preserve or extend islet cell function and/or endogenous insulin secretion for at least about 1, 2, 3, 4, 5, 6, 7, 8, 9, and 10 years after a patient is diagnosed with T1DM. Patient treated by the methods disclosed herein can be continually managed with insulin therapy as needed.

The metabolic status of a patient treated with a therapeutic compounds as disclosed herein can also be detected by measuring fasting blood glucose (glucose-oxidase method), glycosylated haemoglobin A1c (HbA1c) blood levels (Naka K., Japan J. Clin. Lab. Automation 6(suppl.):22, 1981), and triglycerides concentrations (Bucolo and David, Clin. Chem. 19:476-82, 1973). Administration of one or more of the therapeutic agents as disclosed herein can increase HbA1c blood levels after 12-months versus a control group treated conventionally. For example, these patients will have less than about 8%, 8.5%, 7%, 7.5%, 6%, 6.5% or 5% HbA1c levels as compared to a control group. Target levels should be in accordance with the ADA recommendations for HbA1c, i.e., levels of <8% in school age children and <7.5% in adolescents and young adults, with preprandial glucose levels of 90-130 mg/dl (plasma), postprandial levels of <180 mg/dl, and bedtime levels of 90-150 mg/dl. In certain aspects, the HbA1c levels are not associated with significant hypoglycemia.

In particular embodiments of the present disclosure, one or more of the therapeutic agents identified above as promoting the shift of Th1 to Th2 cells is administered to a patient as soon as possible after diagnosis of T1DM. Alternately, one or more of the therapeutic agents is administered after a diagnosis that a patient is at risk for T1DM but prior to the onset of the disease. The one or more identified therapeutic agents can be administered continuously to prevent or protect against the subsequent destruction of islet cells in the patient. In order to prevent any significant reduction in the effectiveness of the therapies of the present disclosure, it may be desirable in some instances to increase the dosage of the therapeutic agent being administered, to subsequently administer one or more additional therapeutic agents identified as promoting the shift of Th1 to Th2 cells, and/or to shift from the use of one of the identified therapeutic agents to another such compound.

Patients in the early stages of or at risk for T1DM can be identified by a number of methods. Because there appears to be a genetic component to T1DM, it is possible in some cases for a physician to identify a patient as being at risk for T1DM based on family history. It is also possible to detect a patient in the early stages of or at risk for T1DM by detecting immune markers in their blood such as antibodies against insulin, islet cells, the enzymes glutamic acid decarboxylase (GAD) and IA2 (also known as ICA512), or other auto-immune antibodies that have been identified as having a correlation with the onset of T1DM. It is now also possible to detect an enhanced risk of T1DM based on a patient having certain genetic markers, which can be readily identified. Several of the primary genetic markers currently relied upon are in the HLA region of chromosome 6. The HLA-DQ locus is a strong single marker of susceptibility to T1DM, particularly among Caucasians. Recent genomic screens have identified numerous other loci that may also contribute to the risk of developing T1DM. Numerous indicators of increased risk for developing T1DM are known or will be identified, and such tests and their proper use will be well-known to those skilled in the art.

Statins have been found to be safe and effective for use in children suffering from hypercholesterolemia. For example, atorvastatin is now approved for the treatment of heterozygous familial hypercholesterolemia (FH) in boys and postmenarchal girls between the ages of 10 and 17 (See, e.g., Athyros et al., Arteriosclerosis 163(1):205-206, 2002; Munoz et al., Circulation 108(17):IV689, 2003). It is indicated as an adjunct to diet to reduce total cholesterol, low-density lipoprotein cholesterol, and apolipoprotein B levels in this population. The recommended starting dose for juveniles with FH is 10 mg/day, which can be increased to a maximum of 20 mg/day in children aged 10-17 years and up to 80 mg/day in adults (and in children with homozygous FH). The safety profile and efficacy of many of the statins have been demonstrated in hundreds of ongoing and completed clinical trials involving tens of thousands of patients. The number of adverse events observed with use of many of the statins is low.

Many of the therapeutic agents useful in the present disclosure, including statins, are capable of inhibiting sterol synthesis in a patient. Inhibition of the synthesis of cholesterol or other sterols, however, may be undesirable, particularly in very young patients where it could interfere with normal development. Therefore, in certain embodiments, the therapeutic agents of the present disclosure are administered to a juvenile in an amount that is below the IC50 for inhibition of sterol synthesis and above the IC50 for treatment of the juvenile for T1DM. Methods for determining the dosages of the therapeutic agents of the present disclosure which will reduce or delay destruction of islet cells while minimizing any side effects of sterol synthesis inhibition will be apparent to those skilled in the art.

As set forth above, the present disclosure describes methods of administering one or more of the therapeutic agents described herein in a therapeutically-effective amount to a juvenile subject at risk for or suffering from T1DM. The therapeutic agent can be administered in a pharmaceutical composition that comprises the compound itself, or a pharmaceutically-acceptable salt, derivative, analog, prodrug, or solvate thereof. The pharmaceutical composition can also comprise a pharmaceutically-acceptable vehicle, diluent, or carrier. The suitability of any particular compound disclosed herein to treat or prevent T1DM in a juvenile patient may be determined by evaluation of its potency and selectivity using literature methods followed by evaluation of its toxicity, absorption, metabolism, and/or pharmacokinetics in a juvenile patient, in accordance with standard pharmaceutical practice.

As used herein, a “pharmaceutically-acceptable salt” is understood to mean a compound formed by the interaction of an acid and a base, the hydrogen atoms of the acid being replaced by the positive ion of the base. Pharmaceutically-acceptable salts, within the scope of this disclosure, include both organic and inorganic types such as, for example, salts formed with ammonia, organic amines, alkali metal hydroxides, alkali metal carbonates, alkali metal bicarbonates, alkali metal hydrides, alkali metal alkoxides, alkaline earth metal hydroxides, alkaline earth metal carbonates, alkaline earth metal hydrides and alkaline earth metal alkoxides. Representative examples of bases that form such base salts include, but are not limited to, ammonia, primary amines such as n-propylamine, n-butylamine, aniline, cyclohexylamine, benzylamine, p-toluidine, ethanolamine and glucamine; secondary amines such as diethylamine, diethanolamine, N-methylglucamine, N-methylaniline, morpholine, pyrrolidine and piperidine; tertiary amines such as triethylamine, triethanolamine, N,N-dimethylaniline, N-ethylpiperidine and N-methylmorpholine; hydroxides such as sodium hydroxide; alkoxides such as sodium ethoxide and potassium methoxide; hydrides such as calcium hydride and sodium hydride; and carbonates such as potassium carbonate and sodium carbonate. Example of non-toxic acid addition salts include but are not limited to potassium, ammonium, hydrochloric, hydrobromic, hydroiodic, sulphate or bisulphate, nitrate, phosphate or hydrogen phosphate, acetate, benzoate, succinate, saccarate, fumarate, maleate, lactate, citrate, tartrate, gluconate, camsylate, methanesulphonate, ethanesulphonate, benzene-sulphonate, p-toluenesulphonate and pamoate salts. The compounds for use in the present disclosure can also provide pharmaceutically acceptable metal salts, in particular non-toxic alkali and alkaline earth metal salts, with bases. Examples include the sodium, potassium, aluminium, calcium, magnesium, zinc and diethanolamine salts. For a review on suitable pharmaceutical salts, see Berge et al, J. Pharm, Sci. 66, 1-19, 1977, incorporated herein by reference.

As used herein, “derivative” refers to chemically modified inhibitors or stimulators that still retain the desired effect or property of the original therapeutic agent. Such derivatives may be derived by the addition, removal, or substitution of one or more chemical moieties on the parent molecule. Such moieties may include, but are not limited to, an element such as a hydrogen or a halide, or a molecular group such as a methyl group. Such a derivative may be prepared by any method known to those of skill in the art. The properties of such derivatives may be assayed for their desired properties by any means known to those of skill in the art. As used herein, “analogs” include structural equivalents or mimetics.

A variety of administration routes are available for delivering the therapeutic agents disclosed herein to a patient in need. The particular route selected will depend upon the particular drug selected, the weight and age of the patient, and the dosage required for therapeutic effect. The pharmaceutical compositions may conveniently be presented in unit dosage form and may be prepared by methods well-known in the art of pharmacy. The therapeutic agents suitable for use in accordance with the present disclosure, and their pharmaceutically acceptable salts, derivatives, analogs, prodrugs, and solvates can be administered alone, but will generally be administered in admixture with a suitable pharmaceutical excipient diluent or carrier selected with regard to the intended route of administration and standard pharmaceutical practice.

The methods of the present disclosure may be practiced using any mode of administration that is medically acceptable, meaning any mode that produces therapeutically effective levels of the active therapeutic agents without causing clinically unacceptable adverse effects. Such modes of administration include, but are not limited to, oral, rectal, topical, nasal, pulmonary, interdermal, or parenteral routes. As used herein, the term “parenteral” includes subcutaneous, intravenous, intramuscular, or infusion routes of administration. Intravenous or intramuscular routes are not particularly suitable for long-term therapy and prophylaxis. Oral administration can be a more convenient route of administration for long-term therapy and prophylaxis. Compositions suitable for oral administration to older juveniles include discrete solid units, such as capsules (including soft gel capsules), tablets, lozenges, multi-particulates, gels, films, or ovules, each containing a predetermined amount of one or more of the compounds, for example statins, disclosed herein. Other compositions include solutions or suspensions in aqueous liquids or non-aqueous liquids such as a syrup, elixir or an emulsion, which may be a more appropriate dosage form for infants and younger juveniles. The dosage forms may contain flavoring or coloring agents, for immediate-release, delayed-release, modified-release, sustained-release, dual-release, controlled-release or pulsatile delivery applications. Such compounds also may be administered via fast dispersing or fast dissolving dosages forms or in the form of a high energy dispersion or as coated particles. Suitable pharmaceutical formulations may be in coated or un-coated form as desired. Such systems can avoid repeated administrations of the compounds disclosed herein, thereby increasing convenience to the subject and the physician, as well as improving patient compliance with the dosage regimen.

In certain embodiments use of a long-term sustained-release implant is a particularly suitable delivery system for juveniles who may find compliance with a dosage regimen difficult. Long-term release, as used herein, means that the implant is constructed and arranged to deliver therapeutic levels of the active ingredient for at least 8-hours, 12-hours, 24 hours, 36 hours, 48 hours, 72 hours, 1 week, 2 weeks, 3 weeks, 4 weeks, 30 days, or 60 days. Methods for producing long-term sustained-release implants are well-known to those of skill in the art. In other embodiments of the present disclosure, compounds disclosed herein are administered continuously to the patient as long as the patient exhibits islet cell function and for as long as there is any concern that the patient will produce an auto-immune response to islet cells if the patient is not appropriately treated.

The appropriate dosages (in single or divided doses) of the therapeutic agents disclosed herein for juveniles can be determined using methods well known to those of skill in the art. The dosages will vary depending on the compound, but in certain embodiments a therapeutically effective amount of a therapeutic agent will be within the range of about 0.001 mg per kg per day (mg/kg/day) to about 20 mg/kg/day, about 0.25 mg/kg/day to about 0.55 mg/kg/day, or about 0.55 mg/kg/day to about 5 mg/kg/day. In other embodiments, a therapeutically effective amount of a therapeutic agent disclosed herein for treatment of an infant is in the range of about 0.1 mg/day to about 20 mg/day, about 1 mg/day to about 10 mg/day, or about 2 mg/day to about 5 mg/day. A therapeutically effective amount of a therapeutic agent disclosed herein for treatment of a pre-pubescent child is in the range of about 0.5 mg/day to about 80 mg/day, about 2 mg/day to about 40 mg/day, or about 5 mg/day to about 20 mg/day. A therapeutically effective amount of a therapeutic agent disclosed herein for treatment of a pubscent or adolescent child is in the range of about 0.5 mg/day to about 120 mg/day, about 5 mg/day to about 80 mg/day, or about 10 mg/day to about 40 mg/day. Dosage may by via single dose, divided daily dose, multiple daily dose, or continuous (chronic) daily dosing for a specified period of time. The physician in any event will determine the actual dosage which will be most suitable for each individual patient, which may vary with the age, weight and response of the particular patient. The above dosages are exemplary of the average case. There can, of course, be individual instances where higher or lower dosage ranges are merited and such are within the scope of this disclosure.

Solid pharmaceutical compositions, e.g., tablets, may contain excipients such as microcrystalline cellulose, lactose, sodium citrate, calcium carbonate, dibasic calcium phosphate, glycine and starch (e.g., corn, potato or tapioca starch), disintegrants such as sodium starch glycollate, croscarmellose sodium and certain complex silicates, and granulation binders such as polyvinylpyrrolidone, hydroxypropylmethyl cellulose (HPMC), hydroxypropylcellulose (HPC), hydroxypropyl methylcellulose acetate succinate (HPMCAS), sucrose, gelatin and acacia. Additionally, lubricating agents such as magnesium stearate, stearic acid, glyceryl behenate and talc may be included. Solid compositions of a similar type may also be employed as fillers in gelatin capsules or HPMC capsules. Example excipients in this regard include lactose, starch, a cellulose, milk sugar or high molecular weight polyethylene glycols. For aqueous suspensions and/or elixirs, the compounds of the present disclosure may be combined with various sweetening or flavouring agents, colouring matter or dyes, with emulsifying and/or suspending agents and with diluents such as water, ethanol, propylene glycol and glycerin, and combinations thereof.

Modified release and pulsatile release dosage forms may contain excipients such as those detailed for immediate release dosage forms together with additional excipients that act as release rate modifiers, these being coated on and/or included in the body of the device. Release rate modifiers include, but are not exclusively limited to, HPMC, HPMCAS, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer, and mixtures thereof. Modified release and pulsatile release dosage forms may contain one or a combination of release rate modifying excipients. Release rate modifying excipients may be present both within the dosage form, i.e., within the matrix, and/or on the dosage form, i.e., upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, or xylitol. The terms dispersing or dissolving as used herein to describe FDDFs are dependent upon the solubility of the drug composition used, i.e., where the drug composition is insoluble a fast dispersing dosage form can be prepared and where the drug composition is soluble a fast dissolving dosage form can be prepared.

The compounds comprising the therapeutic agent described herein that are suitable for use in accordance with the present disclosure can also be administered parenterally, for example, intracavernosally, intravenously, intra-arterially, intraperitoneally, intrathecally, intraventricularly, intraurethrally, intrasternally, intracranially, intramuscularly or subcutaneously, or they may be administered by infusion or needle-free techniques. For such parenteral administration, they are best used in the form of a sterile aqueous solution which may contain other substances, for example, enough salts or glucose to make the solution isotonic with blood. The aqueous solutions should be suitably buffered (e.g., a pH of from 3 to 9 can be used), if necessary. The preparation of suitable parenteral formulations under sterile conditions is readily accomplished by standard pharmaceutical techniques well-known to those skilled in the art.

The following examples are included to demonstrate certain embodiments of the disclosure. It should be appreciated by those of skill in the art that the techniques disclosed in the examples which follow represent techniques discovered by the inventor to function well in the practice of the disclosure, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the disclosure.

### EXAMPLE 1

The non-obese diabetic (NOD) mouse model is the best-studied animal model of autoimmune diabetes. Although the development of diabetes in this model has certain important differences from T1DM which occurs in humans, the NOD mouse has become the standard model for investigating the pathogenesis of autoimmune diabetes, and for evaluating potential therapeutic interventions (Atkinson and Leiter, Nature America 5:601-604, 1999). Spontaneous diabetes occurs in female NOD mice and is preceded by insulitis, a lymphocytic infiltration of the pancreatic islets, which is the major histologic event occurring by 5-8 weeks in the NOD mouse (Chatenoud et al., Proc. Natl. Acad. Sci. 91:123-127, 1994).

In an initial experiment, simvastatin was administered to NOD mice, and resulted in a lower level of glucose during postnatal development (p=0.013, paired, one tailed). The study was not long enough (70 days), however, to detect a significant difference in frank diabetes (glucose >300 mg/dl). The length of the study was also the problem found in the study by Palomer et al., Diabetologia 48:1671-1673 (2005) in which they concluded that atorvastatin has no effect on preventing diabetes. Based on the results of the initial experiment, a more exhaustive study was performed to examine the ability of atorvastatin at 5 mg/kg/daily oral dose or 10 mg/kg/daily oral dose to prevent the development of diabetes in NOD mice. See Key et al., Atorvastatin Prevents Diabetes in NOD Mice, Departments of Pediatrics and Biometry, MUSC The Charles P. Darby Children's Research Institute MUSC Children's Hospital, Abstract PAS Meeting, Washington D.C., May 2005, incorporated herein by reference. In these experiments, 4-week old female NOD mice were obtained from Jackson Laboratories (JAX strain no 001976; NOD/LtJ strain) and maintained in a sterile environment. Drug treatment began at 4-weeks of age, prior to inflammatory disease onset. Under normal conditions, diabetes begins appearing in NOD mice by 12-weeks in untreated animals, with 90-100% of the mice developing the disease by 30-weeks.

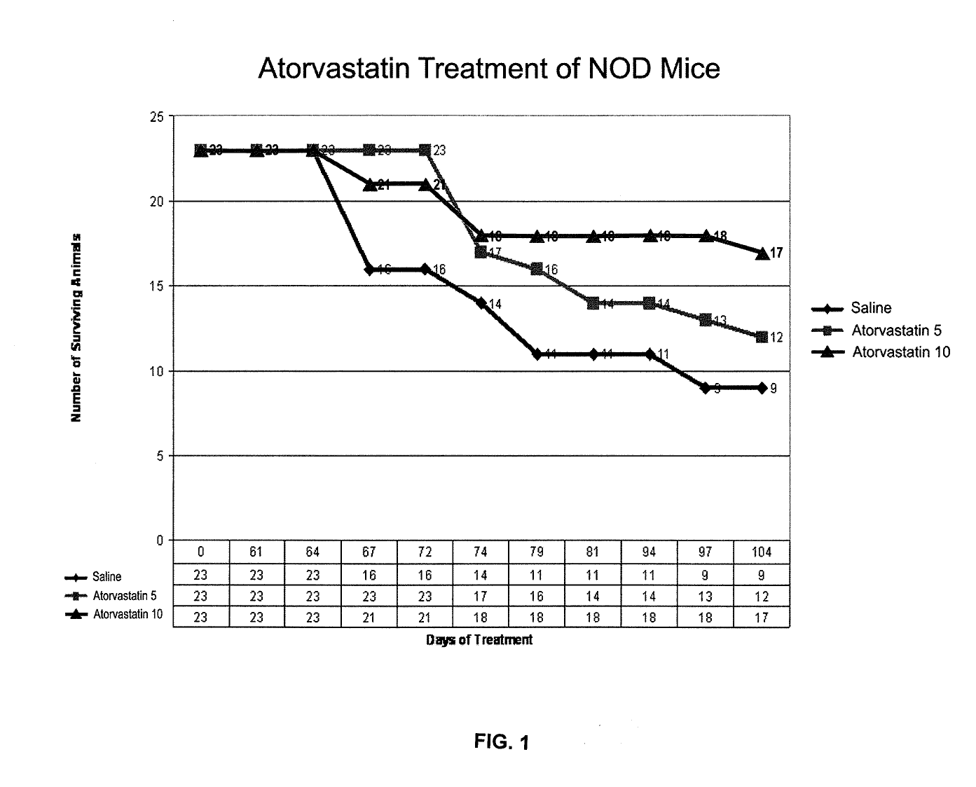

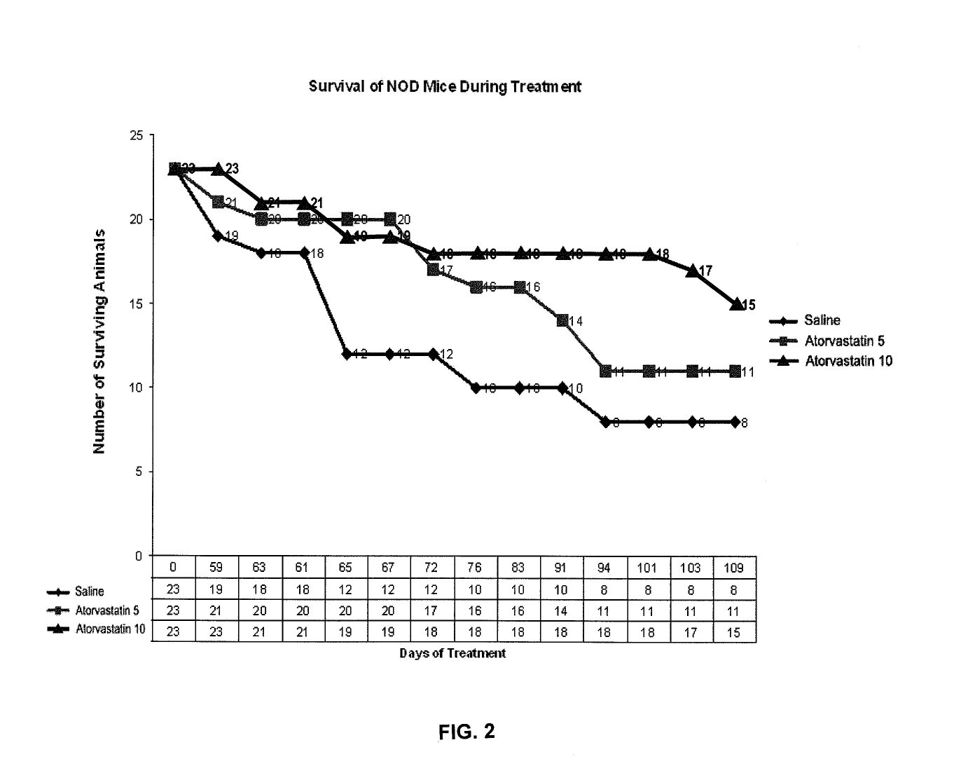

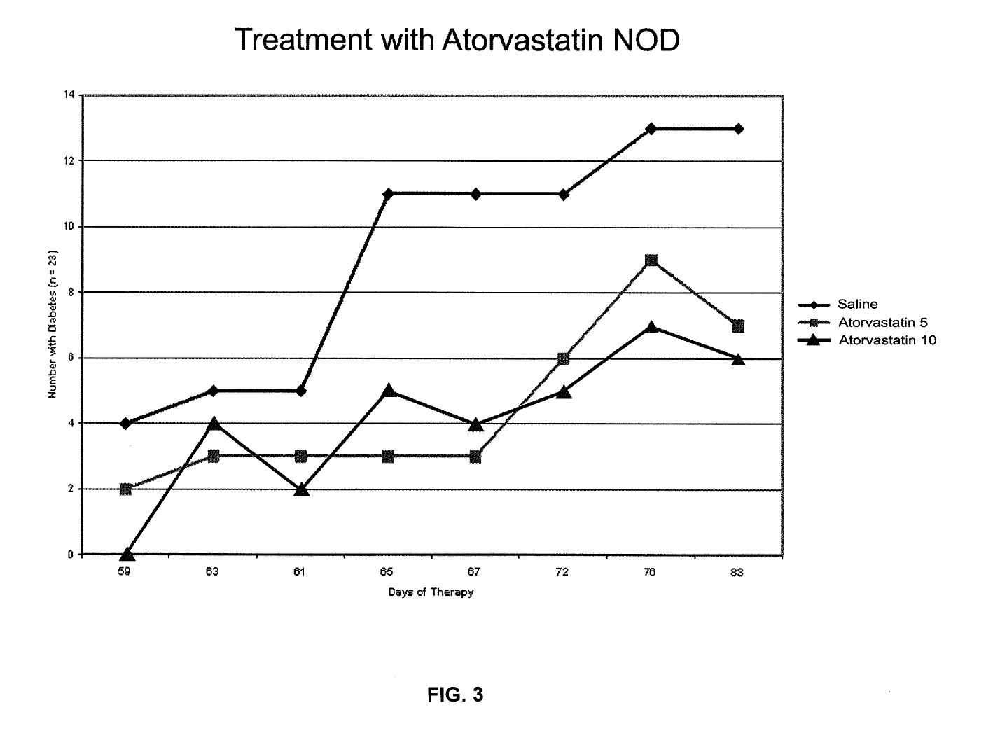

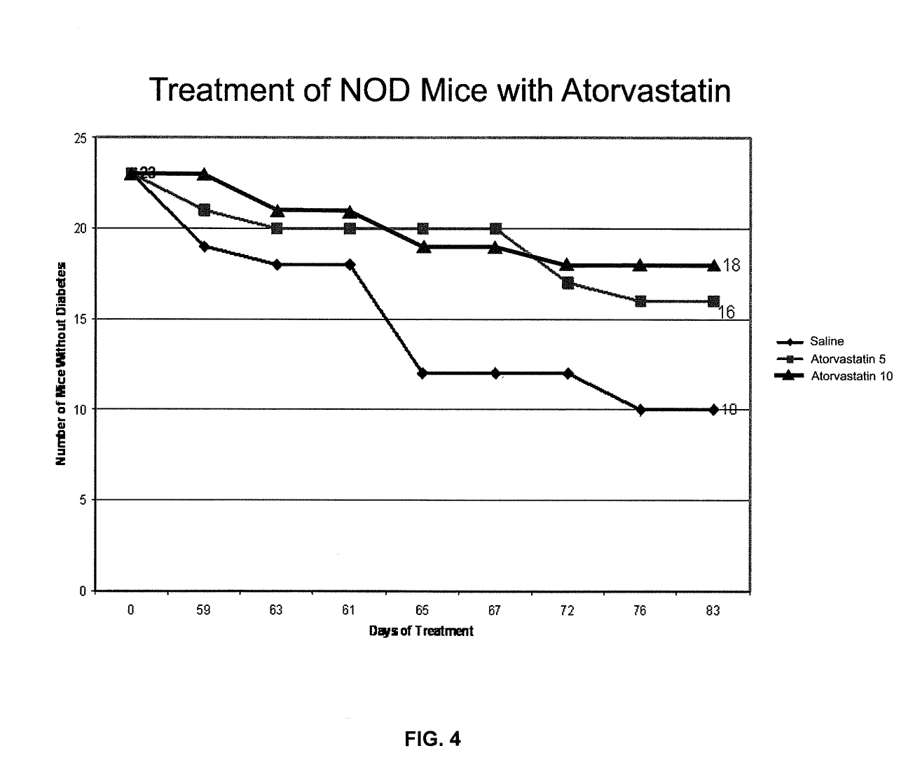

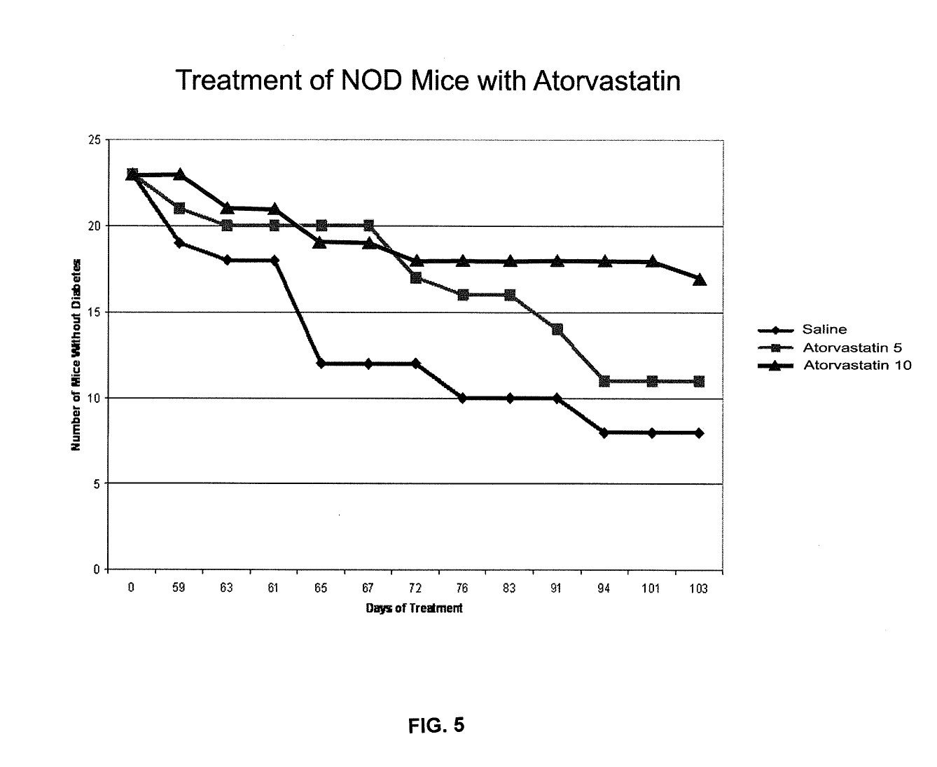

The animals were divided into three groups of 23 animals each. Group 1 was the control and received vehicle only by oral lavage of physiological saline daily. Groups 2 and 3 received atorvastatin calcium (Lipitor®) at a dose of 5 or 10 mg/kg body weight daily, respectively. Blood glucose (BG) was measured in each animal three-times per week. Animals that were found to have two consecutive BG values of over 300 were classified as having diabetes and were sacrificed. Also, two non-diabetic animals from each group were sacrificed on days 50, 70, and 80 for histological and cytokine analyses (not included in FIG. 1). The mean BGs were lower in the 10 mg/kg atorvastatin (182.5 (s.e.=15, n=277, p<0.001)) treatment group compared to the control (Saline=219 (s.e.=11, n=248)). The number of surviving disease-free animals after 104 days of treatment was greater in the groups receiving atorvastatin than in the control group, FIG. 1. The difference between the placebo and 10 mg/kg group was statistically significantly (FIG. 1; log-rank test; p=0.004). This same basic experiment was repeated, with similar numbers of NOD animals surviving disease-free after 109 days of treatment, as shown in FIG. 2. In addition, the number of NOD mice with diabetes treated with placebo and atorvastatin after 83 days of treatment is shown in FIG. 3, while the number of treated NOD mice without diabetes after 83 days and 103 days is shown in FIGS. 4 and 5, respectively. This data suggests that atorvastatin partially protects NOD mice from the development of auto-immune diabetes.

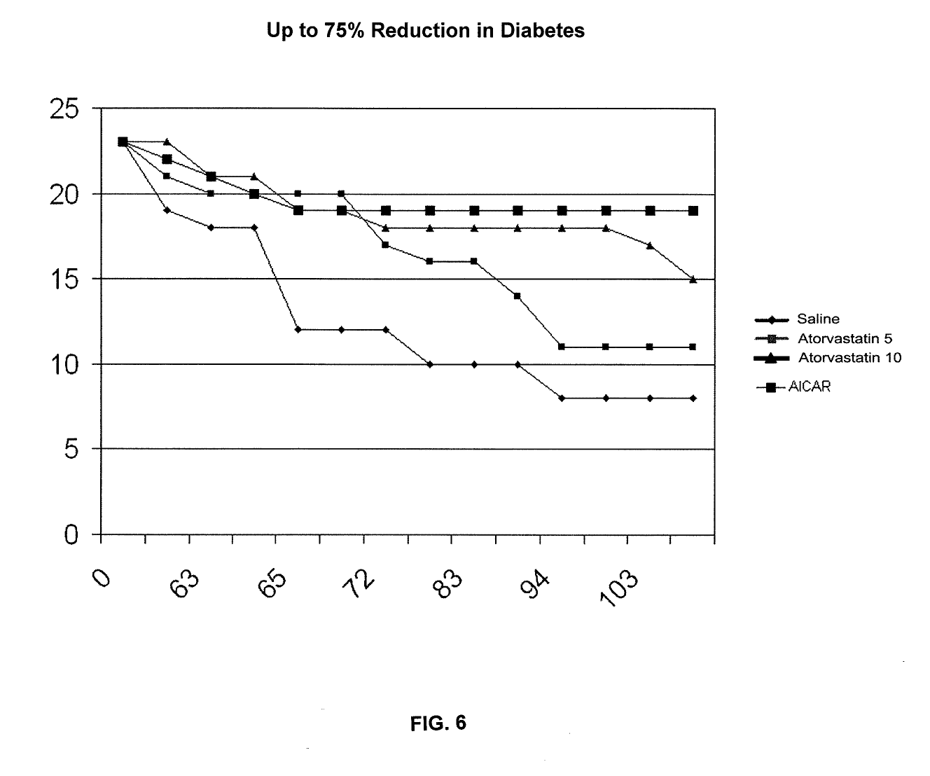

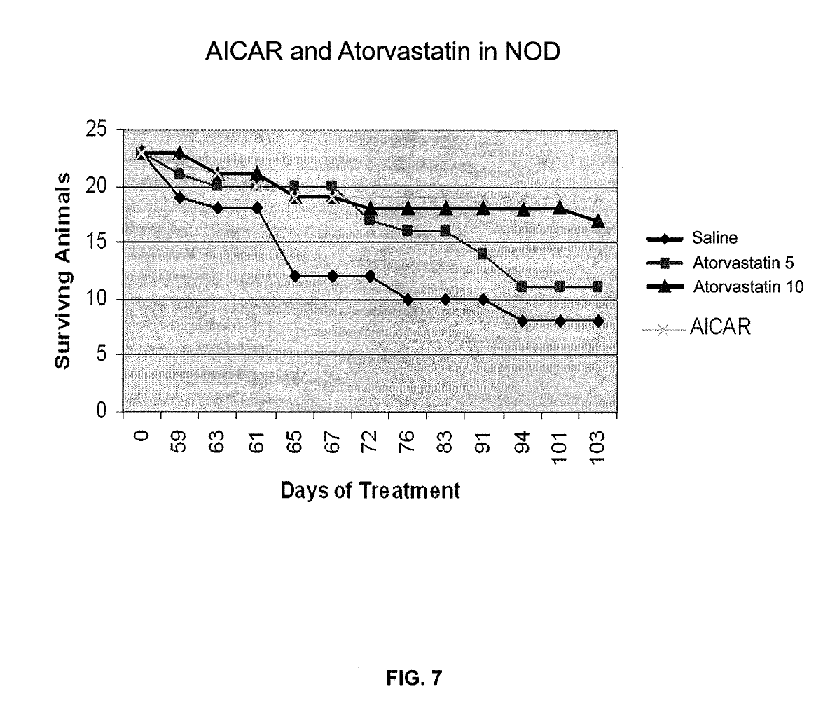

During treatment, atorvastatin resulted in a reduction in the number of animals that developed diabetes (FIG. 1). In addition to statins, AICAR was also administered to NOD mice at 10 mg/kg body weight per day orally. As shown in FIG. 6, while atorvastatin was effective (p<0.004) compared to saline, AICAR was significantly different at p<0.001. FIG. 7 shows the number of surviving disease-free NOD animals after 104 days of treatment with atorvastatin or AICAR. NOD mice treated with AICAR also had a similar reduction in average glucose levels. These data indicate that treatment with statins and AICAR reduces the number of NOD animals with diabetes, and improves the glucose levels in an apparently dose-dependent fashion.

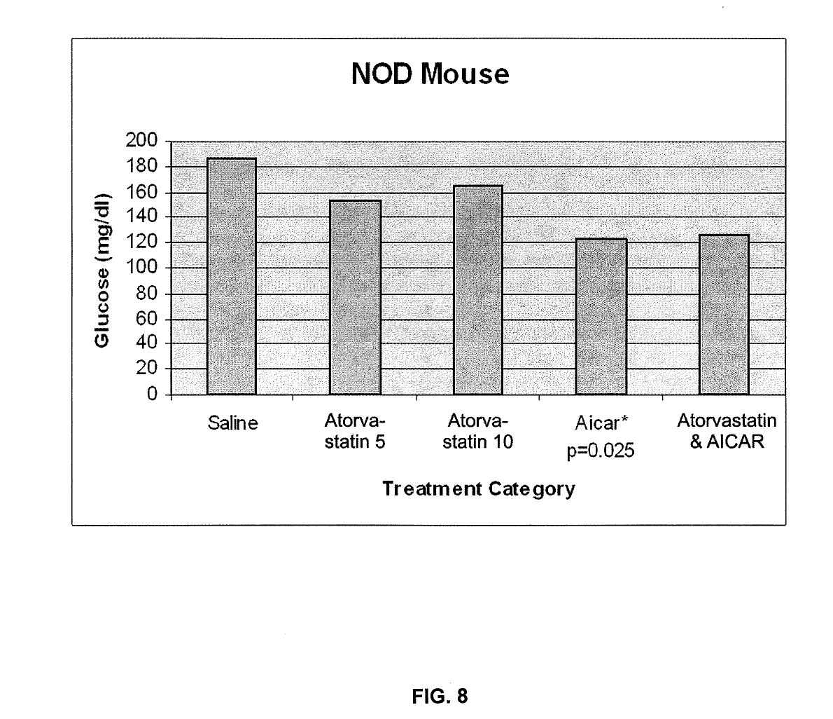

In another experiment that tested the therapeutic effect of atorvastatin calcium (Lipitor®) and AICAR in NOD mice, both individually and in combination, NOD mice were obtained from Toconic (Bomholt, Denmark), and maintained under pathogen-free conditions. Under standard conditions, over 90% of NOD mice will develop diabetes by 30 weeks of age. The NOD mice obtained were divided into five groups of 30 animals each. Group 1 was the control and received vehicle only by oral lavage daily (Saline). Groups 2 and 3 received atorvastatin calcium daily at an oral dose of 5 mg/kg body weight (Lipitor 5) or 10 mg/kg body weight (Lipitor 10). Group 4 received AICAR daily at an oral dose of 0.5 mg/gram (gm) body weight (Aicar). Finally, Group 5 received a combination of atorvastatin calcium daily at an oral dose of 10 mg/kg body weight and AICAR daily at an oral dose of 0.5 mg/gm body weight (L+A). Blood glucose (BG) was measured in each animal on day 59, 61, 63, 65, 67, 72, 76, 83, 91, 94, 101, 103, and 109 after weaning (at 4 weeks). Thus, the age of each animal the days BG was measured was 87, 89, 91, 93, 95, 100, 104, 111, 119, 122, 129, 131, and 137 days, respectively. The results of these measurements are shown in FIG. 8. The mean BG measurements for Groups 2-5 were all lower then for control Group 1. The difference between control Group 1 and Group 4 (Aicar) was statistically significant (p=0.025 by log-rank test).

This data demonstrates that there is a reduction of inflammation with either atorvastatin calcium or AICAR, or a combination thereof, thereby showing that both atorvastatin and AICAR partially protect NOD mice from the development of autoimmune diabetes. These data may reflect a reduction in the number of inflammatory cells that infiltrate the pancreatic tissue, the amount of proinflammatory cytokines that are produced, the amount of iNO Synthetase, and a decrease in cell damage from apoptotic damage.

### EXAMPLE 2

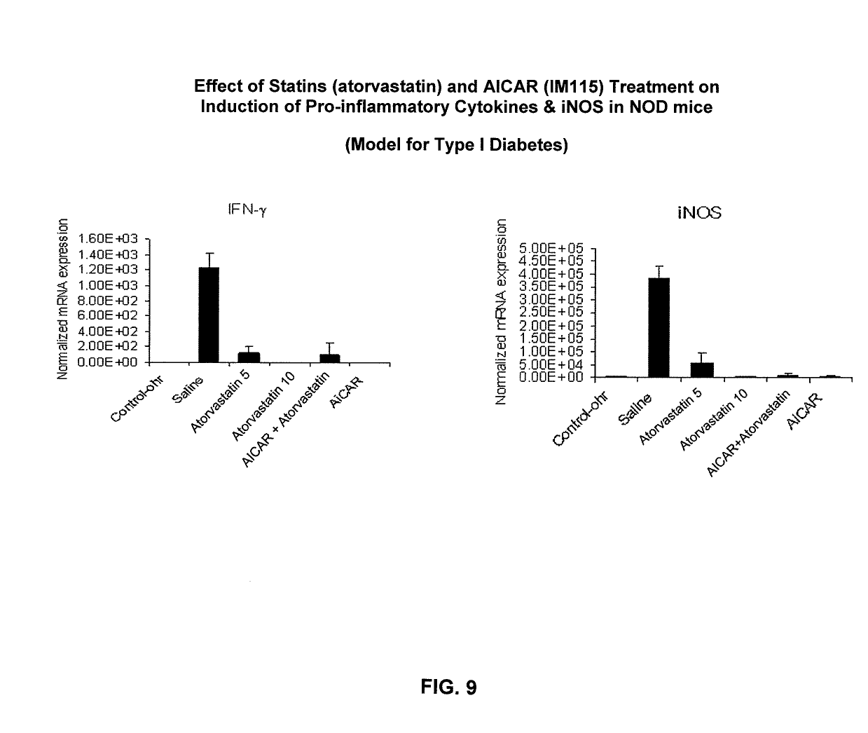

When the animals in Example 1 were sacrificed, the pancreas of the animal was harvested. Healthy control animals (control) were sacrificed on day 0 of treatment for comparison. Immunohistopathology staining of pancreas tissue sections was performed using standard methods of all group animals sacrificed on the same day. Pancreata from each group were analyzed for infiltration of macrophages (ED1) and granulocytes (GR). Immunostaining of tissue sections for ED1(A) and GR1(B) was performed, which demonstrated intense staining in the saline-treated mice for activated macrophages and neutrophils infiltrated into the pancreatic islets. No immunostaining was observed for these marker proteins in atorvastatin treated mice. In addition, animals treated with atorvastatin or AICAR show evidence that inflammatory cells are not entering pancreatic tissue. Immunoblotting by Western blot of the pancreas tissue obtained at the same time point demonstrated increased expression of iNOS and TNF-α protein in saline-treated diabetic mice compared with atorvastatin calcium-treated mice. Finally, expression of pro-inflammatory cytokines (TNF-α and IFN-γ) and iNOS (i.e., mRNA levels) were analyzed by quantitative real-time PCR (FIG. 9). The mRNA expression of iNOS, TNF-α, and IFN-γ was also elevated in saline-treated mice as compared to those mice treated with atorvastatin calcium, AICAR, or a combination of atorvastatin calcium and AICAR (FIG. 9). The remarkable reduction in the cytokines and iNOS appears to come from both a direct effect on the cells in the pancreas, as well as from a relative lack of infiltration into the cells.

These data show that atorvastatin and/or AICAR treatment curtails the infiltration of macrophages and granulocytes, as well as attenuates the induction of TNF-α, IFN-γ and iNOS expression in the pancreas of NOD mice. The anti-inflammatory activity of atorvastatin and AICAR in the pancreas correlated with the lower degree of disease development and higher survival rate of NOD mice treated with atorvastatin and/or AICAR, or a combination of atorvastatin and AICAR, indicating that statins and activators of AMPK are of therapeutic value in NOD mice.

### EXAMPLE 3

In another experiment, simvastatin was administered to NOD mice, and the animals were supplemented with insulin in an attempt to keep the animals from developing diabetic ketoacidosis. The study was designed to examine whether simvastatin at 2 mg/kg/day and 5 mg/kg/day protected islet cells from damage during early phases of diabetes. When the glucose level of the mice in the study rose above 130 mg/dl (upper limit of normal for mice), one unit of insulin was given to the mice once a day using Novolog® (Novo Nordisk). If the glucose level of the mice in the study rose above 150 mg/dl, then one unit of insulin was given to the mice twice a day. The animals were divided into four groups randomly. Group 1 was the control and received oral lavage of physiological saline daily, plus insulin as set forth above. Groups 2 and 3 received simvastatin at a dose of 2 mg/kg/day or 5 mg/kg/day, respectively, along with insulin as set forth above. Group 4 received no treatment with either simvastatin or insulin. Blood glucose (BG) was measured in each animal approximately once a week, unless the glucose level of an animal began to fluctuate, at which time glucose level were measured every other day. This study lasted approximately four months. Table 1 sets forth the data generated in this experiment. The last column of Table 1 is a list of measurements of glucose levels in all animals treated with simvastatin.

TABLE 1

| Group 1 | | Group 2 | | Group 3 | | Group 4 | | | |
|---|---|---|---|---|---|---|---|---|---|
| Saline | | Simv.(2 mg) | | Simv.(5 mg) | | no treatment | | Simvastatin Combined | |
| 146 | 115 | 101 | 206 | 27 | 129 | 103 | 179 | 206 | |
| 80 | 115 | 14 | 148 | 127 | 163 | 94 | 475 | 148 | |
| 17 | 153 | 85 | 102 | 102 | 120 | 147 | 161 | 102 | |
| 5 | 166 | 92 | 119 | 19 | 139 | 148 | 95 | 119 | |
| 116 | 200 | 64 | 122 | 6 | 163 | 84 | 580 | 122 | |
| 95 | 147 | 54 | 124 | 53 | 136 | 144 | 600 | 124 | |
| 16 | 117 | 4 | 150 | 76 | 115 | 141 | 125 | 150 | |
| 146 | 131 | 2 | 90 | 27 | 140 | 42 | 122 | 90 | |
| 80 | 117 | 23 | 161 | 127 | 111 | 105 | 107 | 161 | |
| 17 | 338 | 101 | 160 | 102 | 129 | 108 | 178 | 160 | |
| 5 | 450 | 14 | 140 | 19 | 134 | 73 | 126 | 140 | |
| 116 | 132 | 85 | 133 | 6 | 158 | 51 | 116 | 133 | |
| 95 | 110 | 92 | 128 | 53 | 133 | 52 | 112 | 128 | |
| 16 | 124 | 64 | 180 | 76 | 115 | 139 | 143 | 180 | |
| 146 | 131 | 54 | 141 | 27 | 140 | 119 | 511 | 141 | |
| 80 | 117 | 4 | 133 | 127 | 111 | 120 | 116 | 133 | |
| 17 | 338 | 2 | 116 | 102 | 129 | 121 | 163 | 116 | |
| 5 | 450 | 23 | 147 | 19 | 134 | 65 | 113 | 147 | |
| 116 | 132 | 101 | 160 | 6 | 158 | 45 | 600 | 160 | |
| 95 | 110 | 14 | 140 | 53 | 133 | 103 | 199 | 140 | |
| 16 | 124 | 85 | 133 | 76 | 115 | 147 | 347 | 133 | |
| 146 | 123 | 92 | 128 | 27 | 127 | 148 | 117 | 128 | |
| 80 | 127 | 64 | 180 | 127 | 118 | 113 | 308 | 180 | |
| 17 | 258 | 54 | 141 | 102 | 146 | 114 | 384 | 141 | |
| 5 | 227 | 4 | 133 | 19 | 147 | 141 | 214 | 133 | |
| 116 | 330 | 2 | 116 | 6 | 155 | 42 | 170 | 116 | |
| 95 | 118 | 23 | 147 | 53 | 146 | 105 | 123 | 147 | |
| 16 | 189 | 101 | 183 | 76 | 123 | 108 | 103 | 183 | |
| | | 14 | 171 | | | 73 | 137 | 171 | |
| | | 85 | 137 | | | 51 | 228 | 137 | |
| | | 92 | 122 | | | 52 | 123 | 122 | |
| | | 64 | 150 | | | 139 | 131 | 150 | |
| | | 54 | 143 | | | 120 | 124 | 143 | |
| | | 4 | 155 | | | 65 | 123 | 155 | |
| | | 2 | 118 | | | 103 | 193 | 118 | |
| | | 23 | 214 | | | 147 | 233 | 214 | |
| | | | | | | 148 | 130 | 129 | |
| | | | | | | 113 | 380 | 163 | |
| | | | | | | 114 | 490 | 120 | |
| | | | | | | 141 | 164 | 139 | |
| | | | | | | 42 | 188 | 163 | |
| | | | | | | 105 | 125 | 136 | |
| | | | | | | 108 | 112 | 115 | |
| | | | | | | 73 | 126 | 140 | |
| | | | | | | 51 | 145 | 111 | |
| | | | | | | 52 | 121 | 129 | |
| | | | | | | 139 | 130 | 134 | |
| | | | | | | 120 | 146 | 158 | |
| | | | | | | 65 | 141 | 133 | |
| | 184.6 | | 143.6 | | 134.5 | | 209.7 | 115 | |
| | 102.6 | | 26.5 | | 15.7 | | 143.4 | 140 | |
| | 28.0 | | 36.0 | | 28.0 | | 49.0 | 111 | |
| | | | | | | | | 129 | |
| vs. 1 | 1.000 | | 0.050 | | 0.016 | | 0.376 | 134 | |
| vs. 2 | 0.048 | | 1.000 | | 0.093 | | 0.003 | 158 | |
| vs. 3 | 0.016 | | 0.093 | | 1.000 | | 0.0010 | 133 | |
| vs. 4 | 0.376 | | 0.003 | | 0.0007 | | 1.000 | 115 | |
| N = | 28 | | 36 | | 28 | | 49 | 127 | |
| | | | | | | | | 118 | |
| | | | | | | | | 146 | |
| | | | | | | | | 147 | |
| | | | | | | | | 155 | |
| | | | | | | | | 146 | |
| | | | | | | | | 123 | |
| | | | | | | | | 139.7 | 139.6563 |
| | | | | | | | | 22.7 | 22.74512 |
| | | | | | | | | 0.0014 | 0.029 |
| | | | | | | | | 64 | |

As shown in Table 1, mice treated with insulin and simvastatin had lower levels of glucose than mice that were not treated with simvastatin. In addition, mice treated with simvastatin plus insulin had better glucose control than untreated mice or mice treated with saline. For example, animals treated with only saline plus insulin had an average blood glucose level of 184 mg/dl±102 (in 28 glucose determinations). In the group treated with insulin and 2 mg/kg/day of simvastatin, the average blood glucose level was 143.6±26.5 mg/dl (p=0.05). The lower glucose value and standard deviation suggests that there is greater regulation of the glucose leading to a lower glucose level and less fluctuation, implying that there was protection of islet cell function in these animals. In animals receiving insulin plus 5 mg/kg/day of simvastatin, the mean was 134.5±15.7 mg/dl (p<0.02). In comparison, animals that did not receive any treatment until they were diabetic (over 300) demonstrated a wide range of glucose levels (209.7 mg/dl±143.4 mg/dl). By comparing all animals that were treated with insulin alone (rather then at a starting cutoff of 130 or 300 mg/dl) with all simvastatin-treated animals, a difference in the glucose at a level of p<0.002 was found.

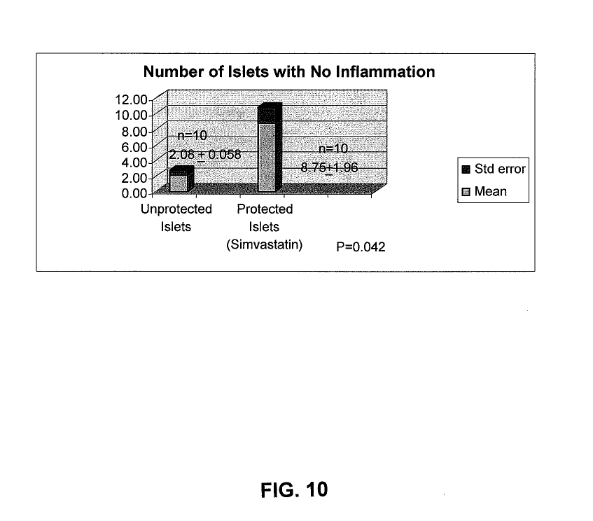

Histological analysis of islet cells in mice treated with simvastatin was also performed. As shown in FIG. 10, lower levels of islet inflammation is found in animals treated with simvastatin. FIG. 10 indicates that islet cells are protected by simvastatin treatment, with over four times as many islet cells without inflammation in mice treated with simvastatin in comparison to unprotected islet cells. It was also found that there appeared to be little variation in the unprotected islet cells. In 6 out of 8 pancrea, no islet cells were found that were undamaged. In the simvastatin group, on the other hand, every pancreas had at least one islet cell that was unharmed. While the histological sections have not been quantified; qualitatively it appears that the insulin production and the number of islet cells showing insulin staining are greater in the islet cells of the animals protected with simvastatin.

Thus, it appears that simvastatin can protects islet cells from inflammation, thereby leading to an increase in insulin production and more stable glucose levels with less glucose variation. While simvastatin was shown to protect islet cells from inflammation and damage from high glucose levels, it has not been shown that simvastatin can stimulate the formation of more islet cells in this very preliminary study. It is possible that insulin producing cells may increase if glucose levels are maintained within the normal range in all animals.

### EXAMPLE 4

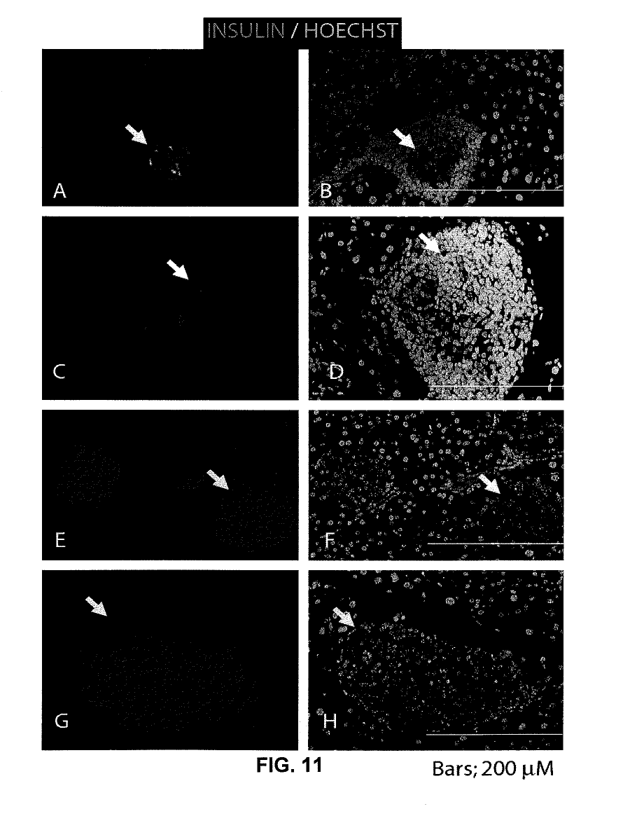

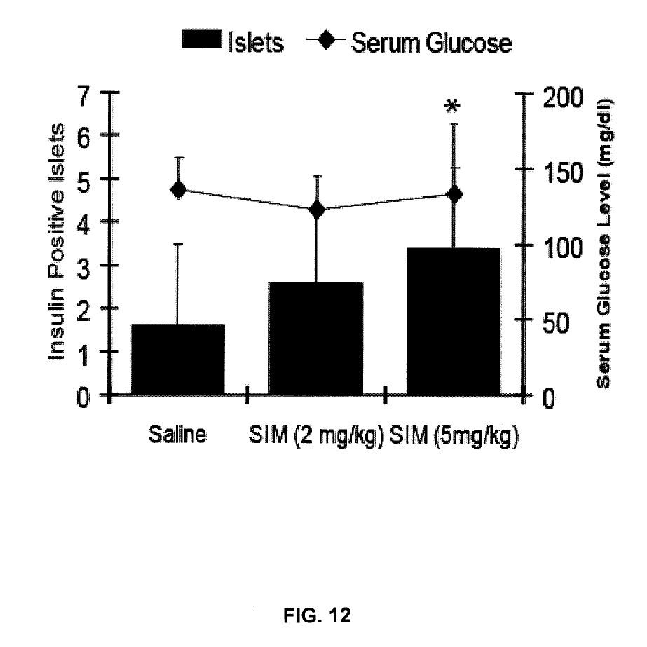

When animals of Example 3 were sacrificed, the pancreas of the animal was harvested and processed for immunohistochemistry and real time PCR analysis. As shown in FIG. 11, simvastatin treatment protected insulin producing islet cells in the pancreas of NOD mice. Immunohistochemistry of pancreas tissue sections was performed using anti-insulin antibodies and counterstained with Hoechst to determine nuclei. Saline treated mice showed loss of insulin producing cells in pancreas islet cells (A and C) with corresponding increase in cellular infiltration (B and D). On the other hand, simvastatin (5 mg/kg) treatment of NOD mice protected insulin producing cells in pancreas islet cells (E and G) via attenuation of cellular Infiltration (F and H). FIG. 12 shows that simvastatin treatment resulted in a significant increase in the number of insulin producing islet cells in the pancreas of NOD mice as compared to those treated with saline. Simvastatin protected pancreas islet cells in a dose dependent manner. Because the glucose level is stable in all three groups of animals, it is not the insulin that is protecting the animals, but rather the simvastatin. Statistical significance is indicated as * p<0.05 versus saline group mice (insulin). RT-PCR analysis showed an increase in insulin message level in the pancreas of NOD mice treated with simvastatin. Pancreas tissues from simvastatin or saline treated NOD mice (n=3/group) were processed for RNA isolation followed by cDNA synthesis using commercially available kits. Real-time PCR was performed using standard kits and insulin specific oligonucleotides. Insulin message was normalized with 18 sRNA expressions in the samples and plotted. Statistical significance is indicated as * p<0.05 and ** p<0.01 versus saline group.

### EXAMPLE 5

T1DM is a T-cell mediated autoimmune disease that is characterized by destruction of the pancreatic islet cells and insulin deficiency. Effective therapy of patients with T1DM necessitates parenteral insulin administration, either by multiple daily injections or by insulin pump. But in many patients, control remains suboptimal and complications develop that severely impact quality of life and shorten life expectancy. At the time of diagnosis, most patients still have significant residual islet cell function, and it has been shown that immunomodulatory intervention can preserve this function and potentially improve short- and long-term blood sugar control. The preliminary studies outlined in Examples 1 and 2 have shown that members of a member of the statin family of drugs, atorvastatin, preserves islet cell function in a mouse model of type 1 diabetes. This research led to a hypothesis that further research with members of the statin class of drugs in combination with intensive insulin therapy may forestall and potentially reverse the destruction of cells in juvenile patients, particularly in juvenile patients who have recently developed T1DM. Based on the above considerations and the known safety profile of statins in adults and children, the following is a proposed clinical trial to study the safety and efficacy of a desired statin (study drug) in preserving cell function in juvenile patients with new-onset T1DM. This clinical trial may also be adapted to study any of the other compounds disclosed herein.

The target study population consists of juvenile subjects between 10-19 years of age with newly diagnosed T1DM as determined by a clinical presentation compatible with this diagnosis and the presence of one or more serum antibodies to islet cell proteins (i.e., anti-GAD65, anti-IA2 or insulin auto-antibodies). In certain aspects, the diagnosis of T1DM will have been made between three and six weeks prior to enrollment in the study, although juvenile patients for whom a longer period of time has passed since diagnosis may also be included in the trial. The calculation for the number of juvenile patients to enroll in the trial will be based on an assumed 5% non-adherence rate during the treatment stage.

Juvenile patients who meet the following criteria will be included in the clinical trial: (1) individuals 10-19 years of age (Tanner Stage 11 or greater) who meet the current Association (ADA) criteria for T1DM; (2) the presence of one or more serum antibodies to islet cell proteins (anti-GAD65, anti-IA2 or insulin auto-antibodies), as assessed in standard practice at each participating institution; (3) diagnosis of T1DM, for example, between 3 and 6 weeks of enrollment; (4) stimulated C-peptide level ≧0.2 pmol/I following a MMTT performed at least 3 weeks after the diagnosis; (5) a parent or legal guardian must provide consent for minor children and patients ages 12 to 17 years old must also provide assent to be in the study; and (6) females of reproductive potential must not plan on conceiving a child during the treatment program, and agree to use a medically accepted form of contraception (e.g., abstinence, barrier method, oral contraceptive, or surgery).

The following criteria will be used to affirmatively exclude individuals from inclusion in the program: (1) individuals currently receiving pharmaceutical products that could interfere or react adversely with the study drug, such as, for example, cyclosporine, fibric acid derivatives, niacin (nicotinic acid), erythromycin, clarythromycin, nefazodone, itraconazole, ketoconazole or protease inhibitors; (2) pregnancy or breast-feeding at the time of eligibility determination; (3) clinical AIDS, ARS or known positive HIV serology; (4) individuals treated with immunosuppressive therapy in the past 12 months; (5) individuals receiving glucocorticoid therapy or therapy other than insulin that is likely to affect glucose homeostasis (such as sulfonylureas, thiazolidinediones, metformin or amylin); (6) individuals with other autoimmune diseases, except autoimmune thyroid disease; (7) individuals with any illness that might complicate diabetes management or preclude treatment with the study drug; (8) transplant recipients; and (9) individuals whose baseline blood test results exceed the limits defined below:
Alanine transaminase (ALT) or aspartate transaminase (AST) greater than twice the upper limit of normal, or creatine phosphokinase (CPK) >3 fold the upper limit of normal;
Total white blood cell count less than 2300/mm³;
Platelet count less than 100,000;
Creatinine >1.5 mg/dl.

Recruitment methods will not involve any restrictions on sociodemographic factors and will be devoid of procedures that may be construed as coercive. Since subjects will be recruited from the pool of newly diagnosed juvenile patients with T1DM, the composition of the subject population will depend on patient sources available to the participating institutions. In order to document the reason for a potential subject's failure to meet eligibility criteria, specific information on each subject signing the informed consent will be entered into a screening log. The eligibility assessment will include: (1) verification that all inclusion/exclusion criteria listed above have been evaluated correctly; (2) evaluation and documentation of relevant medical history, including type 1 diabetes; (3) documentation of medication history; (4) confirmation of diagnosis of T1DM, including confirmation of appropriate islet auto-antibodies; (5) verification that all required information has been documented, and copies of all pertinent reports (e.g., laboratory) have been obtained; and (6) signed and dated informed consent, and assent if applicable. Informed consent will be obtained directly from subjects ≧18 years or from the parent or legal guardian for subjects aged 10 to 17 years. In addition, the assent line on the consent form is to be signed by participants ages 12 to 17 years. Informed consent will be obtained prior to the initiation of any screening procedures performed solely for the purpose of determining study eligibility.

Informed consent may be obtained as follows. The initial consent form will be the Institutional Review Board-approved version of the study site, corresponding to the version of the protocol approved when the screening is initiated. Informed consent will be obtained from the patient or patient's legally authorized representative. A parent or legal guardian of each patient younger than 18 years of age must provide permission for the patient to participate. In addition, assent from participants aged 12 to 17 years may be required. Participants who are 18 years or older will sign a separate consent. The individual responsible for obtaining consent will assure, prior to signing of the informed consent; that the participant has had all questions regarding therapy and the protocol answered. Informed consent will be obtained prior to the initiation of any screening procedures that are performed solely for the purpose of determining eligibility for the study that would not have been performed as part of standard patient care at the respective site. It is the investigator's responsibility to ensure that witnessed informed consent is obtained from the participant or participant's legally authorized representative before participating in an investigational study, after an adequate explanation of the purpose, methods, risks, potential benefits and participant responsibilities of the study. Each participant must be given a copy of the informed consent. The original signed consent must be retained in the institution's records and is subject to review by the NIH, the FDA or representative from another agency that performs the same function, and the Institutional Review Board responsible for the conduct of the institution.

The screening tests outlined in Table 2 may be used to determine eligibility of participants. If all eligibility requirements are met, the subject will be registered and randomized and the following baseline values will be collected: (1) Hemoglobin Ai c, (2) C-peptide levels during a 4-hour MMTT, and (3) blood for cytokine analysis (e.g., C-reactive protein, Interleukin-6 and TNF-α). If eligibility requirements are not met, the subject will not be registered into the study and will continue with standard clinical care.

Following the Baseline visit, the next clinic visit will be scheduled in 14 days (17 days) (Week 2 or W2). Subsequent visits will be scheduled quarterly after baseline assessment, in conjunction with routine diabetes follow-up (within a 14 day window). In addition, the study coordinator will call subjects 14 days after the Week 2 visit and at least monthly thereafter, to assess safety and record any adverse events and concomitant medications. For randomized subjects, the month 12 visit will include a MMTT, after which study medication will be withdrawn. No further protocol-specified clinical evaluations will be made until the 18-month closeout visit, and standard clinical procedures will continue under the direction of the primary physician and diabetes team. This 18-month visit will be conducted solely for the purpose of assessing the consequence of medication withdrawal upon efficacy outcome parameters. Monitoring of adverse events will continue for 30 days beyond the active treatment phase.

TABLE 2

| Months | | Baseline | W2 | M3 | M6 | M9 | M12 | M18 |
|---|---|---|---|---|---|---|---|---|
| Visits | Screening | Randomization | Visit 3 | Visit 4 | Visit 5 | Visit 6 | Visit 7 | Visit 8 |
| Informed Consent | X | | | | | | | |
| Eligibility/Medical History | X | | | | | | | |
| Physical Exam and Vitals | X | | X | X | X | X | X | X |
| Pregnancy Test | X | | | | | | X | |
| CBC with differential | X | | X | X | X | X | X | |
| Platelet Count | X | | X | X | X | X | X | |
| Liver Function Tests | X | | X | X | X | X | X | |
| Electrolytes | X | | X | X | X | X | X | |
| BUN | X | | X | X | X | X | x | |
| Creatine Phosphokinase | X | | X | X | X | X | X | |
| Creatinine | X | | X | X | X | X | X | |
| Glucose | X | | X | X | X | X | X | X |
| Lipid Profile | X | | X | X | X | X | X | |
| Insulin Dose/kg body wt. | | X | X | X | X | X | X | X |
| Blood sugar meter download | | | X | X | X | X | X | X |
| HbAlc | | X | | X | X | X | X | X |
| 4-hr MMTT** | | X | | | | | X | X |
| Concomitant Medications | | X | X | X | X | X | X | |
| Compliance Assessment | | | X | X | X | X | X | |
| Adverse Events | | X | X | X | X | X | | |

*Monthly telephone calls (between visits) will capture compliance, adverse events and concomitant medication use information.
**4-hr MMTT will be done according to the protocol outlined in clinical trials of anti-CD3 monoclonal antibodies (Herold et al., Diabetes 54: 1763-1769, 2005, incorporated herein by reference).

All enrolled subjects will receive standard diabetes care including diabetes education, nutrition counseling, blood glucose monitoring and insulin therapy (either multiple daily insulin injections or insulin pump), and will be monitored for a total of 18 months. The diabetes teams at the participating centers will make adjustments to the diet and insulin regimen based on current American Diabetes Association (ADA) guidelines for management of children and young adults with T1DM (American Diabetes Association. Standards of medical care in diabetes-2006. Diabetes Care 29(Suppl 1):S5-S42, 2006). Like all patients with T1DM, the subjects will be expected to do home glucose monitoring at least four times per day (before meals and at bedtime (HS)), and to bring their meters and log books to clinic visits for analysis. To evaluate the contribution of study drug to metabolic diabetes control, study participants will be asked to document all occurrences of hypoglycemia; and test postprandial and overnight blood glucose levels for 7 days prior to study visits. The active study treatment or the placebo will be administered for 12 months, for example in tablet form.

Each subject will be given a diary with instructions to keep a daily record of the time and day the dose was taken, as well as the insulin dose and injection times. The subject will be asked to bring the diary and dosage containers to the next scheduled clinic visit, to facilitate dose counts. The number of doses taken by the subject will be recorded at each clinic visit. At each clinic visit, the participating institution will ensure that each subject receives a supply of the prescribed dose of study drug sufficient to last until the next scheduled visit. The subject should take the prescribed daily dose at the same time each day. In certain aspects, the time can be at bed-time.

Subjects will be randomized either to a desired daily dose of the study drug, or placebo. Subjects will remain on the given dose for at least 4 weeks, but the dose may be increased to a higher dose for the remainder of the study, unless significant side effects occur. If significant side effects occur at the higher dosage, the dose may be reduced to the initial dose, or treatment may be terminated. Certain of the potential therapeutic drugs identified herein have been associated with liver toxicity, which can be monitored in patients. Doses also may be adjusted due to toxicity effects, such as Grade 2 laboratory toxicity or Grade 2 clinical toxicity, as described herein. Markers for laboratory toxicity are set forth below in Table 3.

TABLE 3
Laboratory Markers of Toxicity

| | Grade 0 | Grade 1 | Grade 2 | Grade 3 |
|---|---|---|---|---|
| | No significant | Mild change | Moderate change | Significant change |
| | change | | | |
| AST | Within normal | 2-3 × ULN, normal | 3-5 × ULN, normal | >5 × ULN |
| | range | on retest | on retest | |
| ALT | Within normal | 2-3 × ULN, normal | 3-5 × ULN, normal | >5 × ULN |
| | range | on retesting | on retest | |
| CK | Within normal | >3 × but <10 × ULN, | >10 × ULN, normal | >10 × ULN with |
| | range | normal on retest | on retest | muscle symptoms |
| Hb | Within normal | <10% decline from | 11-20% decline | >20% decline from |
| | range | baseline | from baseline | baseline |
| Hct | Within normal | <10% decline from | 11-20% decline | >20% decline from |
| | range | baseline | from baseline | baseline |
| Platelets | Within normal | 35,000-50,000/mm³ | 15,000-35,000/mm³ | <15,000/mm³ |
| | range | | | |
| Total | No change or | <25% decline from | 25-50% decline | >50% decline from |
| cholesterol | increase | baseline | from baseline | baseline |
| LDL- | No change or | <25% decline from | 25-50% decline | >50% decline from |
| cholesterol | increase | baseline | from baseline | baseline |

Further clinical markers of toxicity are set forth below in Table 4. If a Grade 2 laboratory toxicity is observed at a particular dose the subject may be removed from the study therapy, and if the toxicity is severe enough with the study drug, the study therapy could be temporarily or permanently discontinued. The test(s) resulting in the abnormal laboratory result(s) may be repeated within 3 days. If the toxicity has decreased to a Grade 1 (2-3 times normal) or less, the treatment dose may be reduced. If toxicity has not decreased to a Grade 1 or less, laboratory test(s) may be repeated on day 7 (and days 10 and 14 if necessary). If the Grade 2 toxicity does not resolve to a Grade 1 or less within 14 days from onset, the subject may be removed from the study therapy. If the toxicity decreases to Grade 1 or less, treatment may be resumed. The safety studies will be repeated weekly for two additional weeks. If there is no additional rise to a Grade 2 toxicity, then the dose may remain the same. The study therapy may continue at the reduced dose for the duration of the study unless a second Grade 2 or higher laboratory toxicity is observed in which case, in the absence of any other explanation for the rise, the subject may be removed from the study therapy.

TABLE 4
Clinical Markers of Toxicity

| Adverse Events | Grade 0 | Grade 1 (mild) | Grade 2 (moderate) | Grade 3 (severe) |
|---|---|---|---|---|
| Body as a whole | | | | |
| Fever | None | Temp. 101-102° F. | Temp 103-104° F. or | Temp >104° F. or |
| | | or 38.3-39.4° C. | 39.4-40.6° C., no | 40.6° C. for >48 |
| | | | infection | hrs, w/o infection |
| Fatigue/malaise | None | In bed <25% | In bed 25-50% | Unable to get out |
| | | waking hours | waking hours | of bed for 24 |
| | | | | hours |
| Weight loss | None | Less than 5% | 5-10% body weight | >10% body weight |
| | | body weight | | |
| Cardiovascular | | | | |
| Palpitation | None | Requires no | Requires drug | Requires |
| | | therapy | therapy | monitoring |
| Chest pain | None | Requires no drug | Requires non- | Requires |
| | | therapy | narcotic analgesics | hospitalization |
| Dermatological | | | | |
| Skin Rash | None | Scattered, | Needs therapy for | Exfoliating lesions |
| | | transient, needs | relief | requiring |
| | | no drug therapy | | immediate |
| | | | | medical attention |
| Itching | None | Needs no therapy | Needs antihistamine | Persistent and |
| | | | or other therapy | non-responsive to |
| | | | | therapy |
| Gastrointestinal | | | | |
| Abdominal pain | None | Transient, needs | Requires medical | Requires |
| | | no drug therapy | attention and drug | hospitalization |
| | | | therapy | |
| Constipation | None | Transient, needs | Requires | Requires invasive |
| | | no therapy | laxatives/enemas | diagnostic studies |
| Diarrhea | None | Needs no drug | Needs dietary | Requires |
| | | therapy | change and therapy | intravenous fluid |
| | | | | therapy |
| Loss of appetite | None | No weight loss | Associated with 5- | Requires |
| | | | 10% weight loss | intravenous or |
| | | | | tube feedings |
| Nausea/vomiting | None | Transient, needs | Persistent, needs | Require |
| | | no therapy | medical attention and | hospitalization |
| | | | drug therapy | |
| Musculo-skeletal | | | | |
| Myalgia | None | Activity normal | Unable to carry-out | Unable to get out |
| | | | usual levels of | of bed |
| | | | activity | |
| Joint pain/swelling | None | Normal activity | Unable to carry-out | Unable to get out |
| | | | usual levels of | of bed |
| | | | activity | |
| Neurological | | | | |
| Headache | None | Requires no drug | Requires non- | Requires |
| | | therapy | narcotic drug therapy | hospitalization |
| Sleep | None | Requires no drug | Requires sedatives | Requires |
| Disturbances | | therapy | | neurological |
| | | | | evaluation |

Patient should be treated according to an intensive diabetes management protocol and followed by the diabetes teams at the institution where the clinical trial is conducted. Decisions concerning diabetes care, including insulin dose adjustments, diet modifications, and insulin regimens will be made based upon a review of each patient's self-monitoring results, HbA1c and lifestyle considerations. For example, HbA1c may be assessed every three months to evaluate metabolic control. The goal of treatment may be to maintain the HbA1c level as close to as normal as possible, without frequent occurrence of hypoglycemia. Target levels should be in accordance with the ADA recommendations, with HbA1c levels of <8% in school age children and <7.5% in adolescents and young adults, with preprandial glucose levels of 90-130 mg/dl (plasma), postprandial levels of <180 mg/dl, and bedtime levels of 90-150 mg/dl. A goal of therapy in this age group is a HbA1c of <8% without significant hypoglycemia. A sufficient number of daily injections of short- and long-acting insulin, or insulin pump therapy, may be used to achieve these glycemic goals. Patients should be expected to do home glucose monitoring at least four times per day (before meals and HS), as well as postprandia and overnight testing for one week prior to study visits and whenever deemed necessary to achieve glycemic targets. Therapeutic decisions will be based solely on clinical considerations and not on participation in the study. Study visits should be coordinated with each patient's regular clinic visits to minimize inconvenience for the participants, encourage participation, and facilitate coordination of care.

If a significantly higher proportion of patients with preserved C-peptide is observed in the treated group after 12 months of daily treatment, it would indicate that the study drug has a measurable salutary effect on islet cell function. This is based on the assumption that, in the context of optimal diabetes management, the decline in C-peptide will be sufficient within the first 12 months of diagnosis to detect a difference between the active and control groups. Torn, et al. have suggested a two year follow up period to evaluate the decline from baseline in C-peptide for newly diagnosed T1DM, based on their random C-peptide data in adults (Torn et al., J Clin Endocrinol Metab. 85:4619-4623, 2000). But Steele and colleagues have demonstrated that, despite intensive diabetes management, children and adolescents with new-onset T1DM show a constant rate of decline in islet cell function from the time of diagnosis, as assessed by C-peptide AUC in response to a MMTT (Steele et al., Diabetes 53(2):426-433, 2004). Recently, this finding has been corroborated by a number of studies (Herold et al., New Eng J Med. 346:1692-1698, 2002; Saudek et al., Rev Diabetic Stud. 1:80-88, 2004; Keymeulen et al., N Engl J Med. 352:2598-2608, 2005). This assessment may be further facilitated by the addition of a MMTT performed at 18 months (six months into active study drug withdrawal). Data collected at 18-months will provide estimates of islet cell preservation (MMTT and insulin use) and metabolic diabetes control (HbA1c levels and glucose meter downloads). This will provide valuable insight into the disease progression post-treatment. Finally, this study should be an early attempt to evaluate both the safety and efficacy of the drug studied in a juvenile patient population. It is also possible that the study drug ultimately may need to be coupled with other agents that work via alternate mechanisms for the most effective treatment of this class of patients.

Other aims of the clinical trial will include evaluating the effects of the study drug on C-peptide production and metabolic control, for example by measuring one or more of the following: (1) a 2-hour C-peptide AUC in response to the MMTT at baseline vs. 12 months; (2) a 2- and 4-hour C-peptide AUC in response to the MMTT at 18 months (after a 6-month washout); (3) levels of HbA1c at 3, 6, 9, 12 and 18 months after the initiation of treatment; (4) mean daily insulin dose per kg body weight for the 2 weeks preceding each scheduled study visit; (5) blood glucose control as determined from home glucose meter downloads for the 2 weeks preceding the visit, e.g., the mean blood glucose (BG), number of preprandial BG>160 mg/dl or <70 mg/dl, and postprandial BG>200 mg/dl; and (6) the number of episodes of hypoglycemia requiring any treatment and severe hypoglycemic events, defined by the need for treatment with glucagon, the need for a third party to resolve a hypoglycemic episode, loss of consciousness, or seizure. In addition, medication compliance with the study drug may be measured by drug accountability logs, and the effect of the study drug on cytokine mediators of autoimmunity (e.g. c-reactive protein, interleukin-6, TNFα) may be evaluated.

The primary endpoint of this clinical trial will be in accord with an American Diabetes Association workshop (Palmer et al., Diabetes 53:250-264, 2004, incorporated herein by reference) and TrialNet consensus guidelines for new onset T1DM studies, following review of other new onset T1DM trials, (Herold et al., New Eng J Med. 346:1692-1698, 2002; Allen et al., Diabetes Care 22:1703-1707, 1999; Sklyer et al., Journal of Diabetes & its Complications 6:77-88, 1992; Dupre and Kolb, Diabetes 37:1574-82, 1988), and extensive discussion by the TrialNet steering committee. Other clinical outcome measures of efficacy will include insulin use, HbA1c, and blood glucose levels, all of which support the primary analysis. A list of adverse events closely related to the study drug will be selected as the major safety endpoint. Any information on the dosing of the study drug used in children, as well as the safety spectrum, will be carefully evaluated. Nonetheless, in addition to known drug toxicities, juvenile T1DM patients may be subject to specific adverse events related to use of the study drug in this vulnerable population (e.g., renal insult, cytokine release syndrome, lymphoproliferative disease, opportunistic infection), particularly in younger children. Therefore, insulin requirements and the occurrence of hypoglycemia as well as postprandial hyperglycemia will be frequently evaluated in clinical assessments.

During the clinical trial, the safety of active treatment will be monitored and assessed by routine physical exams, collection of adverse event reports, and by tracking the following laboratory parameters in each patient: creatine phosphokinase (CPK), liver function tests, urinalysis, lipid profile, complete blood count (CBC), blood urea nitrogen (BUN), serum creatinine and electrolytes (sodium, potassium, chloride, bicarbonate). Since T1DM represents a chronic disease with well-defined metabolic consequences, the potential for interaction between diabetes therapies and the study drug may be evaluated. For example, insulin requirements, as well as the occurrence of hypoglycemia and acute and chronic metabolic decompensation, may be monitored in the treated patients.

### EXAMPLE 6

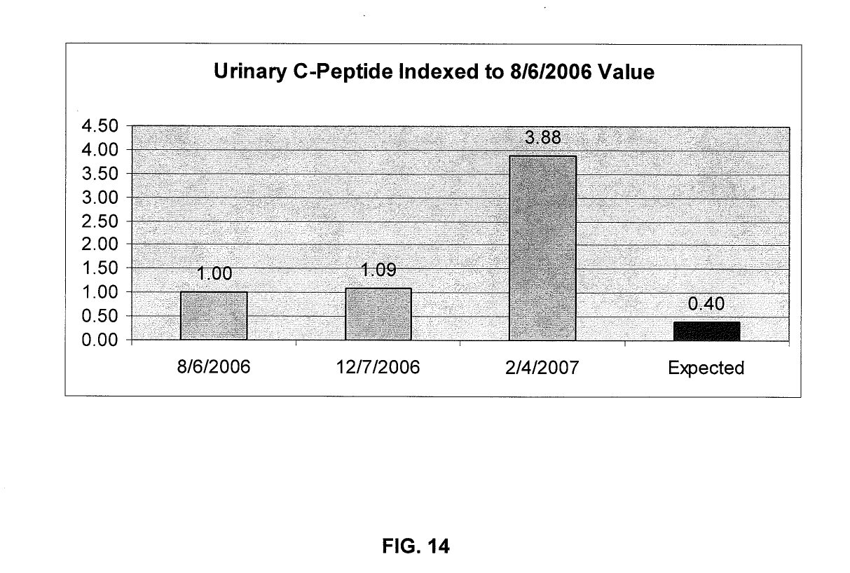

Two juvenile patients newly diagnosed with T1DM were treated with a statin in order to evaluate whether the diabetic condition in the juvenile patient can be stabilized or reversed by statin treatment. An 11-year-old human female patient who was positive for insulin antibodies was identified as having new onset T1DM. FIG. 14 shows the progression of insulin over 6 months of treatment with atorvastatin calcium (Lipitor®) in the patient. The patient was treated with 10 mg/day for the first two weeks after diagnosis, and 20 mg/day thereafter. As shown in FIG. 14, a marked increase in C-peptide was seen in the patient over a short (six month) period of time. The patient elected not to continue treatment with atorvastatin calcium.

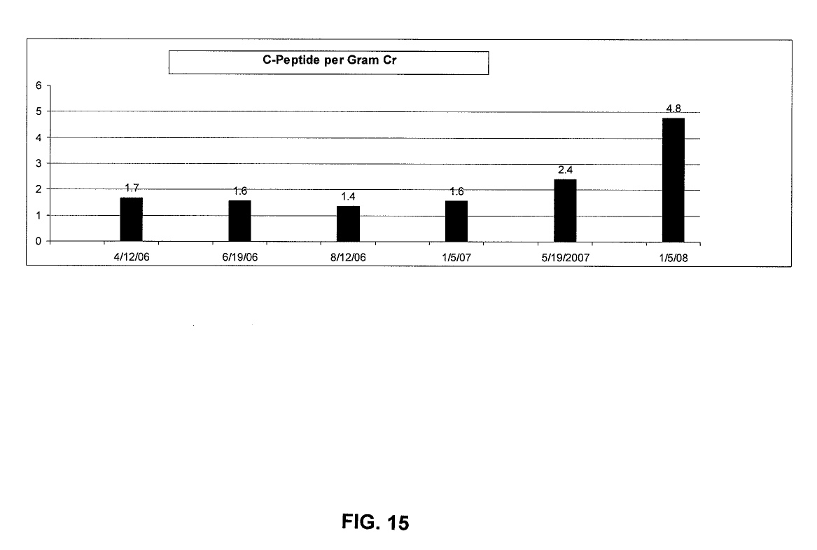

A 17-year-old human male patient who was positive for insulin antibodies was identified as having new onset T1DM. The patient began treatment with 20 mg/day atorvastatin calcium (Lipitor®) at the time (the day of) diagnosis of T1DM. The patient has been maintained on this therapeutic dose. The patient's initial C-peptide test result was 35.8 units. After 50 days of treatment, the patient's C-peptide levels rose 39.25% to 49.85 units, indicating an improvement in islet cell function during the treatment period. FIG. 15 is a graph showing the progression of C-peptide in this patient. As seen over a period of 24 months, the insulin has increased 240% over the original amount of C-peptide (and hence insulin) compared to the time of diagnosis. Normally, within 12 months after diagnosis a child would have been expected to drop from 100% of the insulin the patient started with to 14.0%±7% (s.d.) of the insulin the child started with. This suggests that there is a net increase in this patient of 600% of the amount of insulin that would have been expected, so that rather than losing insulin production as has been demonstrated in Pasquali et al., Diabete Metab. 13:44-51 (1987), the insulin production has substantially increased. Time 0 was 100% at Feb. 15, 2007.

The data shown in FIGS. 14 and 15 suggest that the use of atorvastatin is capable of protecting islet cells and precursors. Therefore, treatment with a statin will reduce or eliminate the expected loss of insulin production, and may stabilize or reverse the diabetic condition in juvenile patient newly diagnosed with T1DM, or at least protect and reduce inflammation in the islets that were undamaged at the time of diagnosis. Given the animal and human data disclosed herein, it appears likely that treatment with a statin allows islet cells to regenerate during treatment, although it is also possible that reduced inflammation resulting from statin treatment allows surviving islet cells to increase insulin production. Since there is now a concern about the viability of islet cell transplant due to damage of the islets over time, this strategy may be a competing, less invasive, and safer strategy for preventing and treating diabetes.

All of the compositions and methods disclosed and claimed herein can be made and executed without undue experimentation in light of the present disclosure. While the compositions and methods of this disclosure have been described in terms of certain embodiments, it will be apparent to those of skill in the art that variations may be applied to the compositions and/or methods and in the steps or in the sequence of steps of the methods described herein without departing from the concept, spirit and scope of the disclosure. More specifically, it will be apparent that certain agents that are chemically or physiologically related may be substituted for the agents described herein while the same or similar results would be achieved. All such similar substitutes and modifications apparent to those skilled in the art are deemed to be within the spirit, scope and concept of the disclosure as defined by the appended claims.

1. A method of treating type 1 diabetes mellitus in a juvenile patient in need of treatment comprising:
(a) identifying a juvenile patient diagnosed with type 1 diabetes mellitus with functioning islet cells, and
(b) administering one or more therapeutic agents to the patient in an amount sufficient to maintain or increase the function of the islet cells in the patient,
wherein the therapeutic agents are selected from the group consisting of an inhibitor of mevalonate synthesis, an inhibitor of 3-hydroxy-3-methylglutaryl coenzyme A (HMG-CoA) reductase, an inducer of AMP protein kinase (AMPK) activity, an inhibitor of dual peroxisome proliferators activated receptor (PPAR) activity, an inhibitor of mevalonic-acid pyrophosphate decarboxylase, an inhibitor of the conversion of isopententyl pyrophosphate (IPP) to farnesyl pyrophosphate (FPP), an inhibitor of the isoprenylation of proteins, an inhibitor of the induction of NF-kβ, an inhibitor of the farnesylation of Ras, an inhibitor of cAMP phosphodiesterase, an antioxidant that blocks LPS- and cytokine-induced production of NO, an enhancer of intracellular levels of cAMP, and any combinations thereof.

2. The method of claim 1, wherein the therapeutic agent is an inhibitor of 3-hydroxy-3-methylglutaryl coenzyme A (HMG-CoA) reductase.

3. The method of claim 2, wherein the inhibitor of HMG-CoA reductase is a statin or a pharmaceutically-acceptable salt, derivative, analog, prodrug, or solvate thereof.

4. The method of claim 3, wherein the statin is selected from the group consisting of lovastatin, mevastatin, atorvastatin, fluvastatin, cerivastatin, pitavastatin, pravastatin, rosuvastatin and simvastatin.

5. The method of claim 1, wherein the function of the islet cells in the patient is maintained.

6. The method of claim 1, wherein the function of the islet cells in the patient increases.

7. A method of treating type 1 diabetes mellitus in a juvenile patient in need of treatment comprising:
(a) identifying a juvenile patient diagnosed with type 1 diabetes mellitus with endogenous insulin secretion, and
(b) administering one or more therapeutic agents to the patient in an amount sufficient to maintain or increase the endogenous insulin secretion in the patient,
wherein the therapeutic agents are selected from the group consisting of an inhibitor of mevalonate synthesis, an inhibitor of 3-hydroxy-3-methylglutaryl coenzyme A (HMG-CoA) reductase, an inducer of AMP protein kinase (AMPK) activity, an inhibitor of dual peroxisome proliferators activated receptor (PPAR) activity, an inhibitor of mevalonic-acid pyrophosphate decarboxylase, an inhibitor of the conversion of isopententyl pyrophosphate (IPP) to farnesyl pyrophosphate (FPP), an inhibitor of the isoprenylation of proteins, an inhibitor of the induction of NF-kβ, an inhibitor of the farnesylation of Ras, an inhibitor of cAMP phosphodiesterase, an antioxidant that blocks LPS- and cytokine-induced production of NO, an enhancer of intracellular levels of cAMP, and any combinations thereof.

8. The method of claim 7, wherein the endogenous insulin secretion in the patient is maintained.

9. The method of claim 7, wherein the endogenous insulin secretion in the patient increases.

10. A method of prolonging the honeymoon period of type 1 diabetes mellitus in a juvenile patient in need thereof comprising:
(a) identifying a juvenile patient in the honeymoon period of type 1 diabetes mellitus, and
(b) administering one or more therapeutic agents to the patient, wherein the one or more therapeutic agents are selected from the group consisting of an inhibitor of mevalonate synthesis, an inhibitor of 3-hydroxy-3-methylglutaryl coenzyme A (HMG-CoA) reductase, an inducer of AMP protein kinase (AMPK) activity, an inhibitor of dual peroxisome proliferators activated receptor (PPAR) activity, an inhibitor of mevalonic-acid pyrophosphate decarboxylase, an inhibitor of the conversion of isopententyl pyrophosphate (IPP) to farnesyl pyrophosphate (FPP), an inhibitor of the isoprenylation of proteins, an inhibitor of the induction of NF-kβ, an inhibitor of the farnesylation of Ras, an inhibitor of cAMP phosphodiesterase, an antioxidant that blocks LPS- and cytokine-induced production of NO, an enhancer of intracellular levels of cAMP, and any combinations thereof,
wherein the administration of the one or more therapeutic agents results in a prolonged honeymoon period in the juvenile patient.

11. The method of claim 10, wherein the juvenile patient in the honeymoon period requires less than 0.5 U/kg/day of insulin.

12. The method of claim 10, wherein the juvenile patient in the honeymoon period has a hemoglobin A1c level equal to or less than 6%.

13. The method of claim 12, wherein the juvenile patient requires less than 0.5 U/kg/day of insulin.

14. A method of preventing type 1 diabetes mellitus in a juvenile at risk of developing type 1 diabetes mellitus comprising:
(a) identifying a juvenile patient at risk for developing type 1 diabetes mellitus, and
(b) administering one or more therapeutic agents to the patient in an amount sufficient to prevent the onset of type 1 diabetes mellitus in the patient,
wherein the therapeutic agents are selected from the group consisting of an inhibitor of mevalonate synthesis, an inhibitor of 3-hydroxy-3-methylglutaryl coenzyme A (HMG-CoA) reductase, an inducer of AMP protein kinase (AMPK) activity, an inhibitor of dual peroxisome proliferators activated receptor (PPAR) activity, an inhibitor of mevalonic-acid pyrophosphate decarboxylase, an inhibitor of the conversion of isopententyl pyrophosphate (IPP) to farnesyl pyrophosphate (FPP), an inhibitor of the isoprenylation of proteins, an inhibitor of the induction of NF-kβ, an inhibitor of the farnesylation of Ras, an inhibitor of cAMP phosphodiesterase, an antioxidant that blocks LPS- and cytokine-induced production of NO, and an enhancer of intracellular levels of cAMP, and any combinations thereof.

15. The method of claim 14, wherein the prevention of type 1 diabetes mellitus in the patient in primary.

16. The method of claim 14, wherein the prevention of type 1 diabetes mellitus in the patient in secondary.

17. A method of treating type 1 diabetes mellitus in a juvenile patient in need of treatment comprising administering one or more therapeutic agents to the patient in an amount sufficient to increase the C-peptide level of the patient after at least six months as compared to the C-peptide level of the patient prior to treatment, wherein the therapeutic agents are selected from the group consisting of an inhibitor of mevalonate synthesis, an inhibitor of 3-hydroxy-3-methylglutaryl coenzyme A (HMG-CoA) reductase, an inducer of AMP protein kinase (AMPK) activity, an inhibitor of dual peroxisome proliferators activated receptor (PPAR) activity, an inhibitor of mevalonic-acid pyrophosphate decarboxylase, an inhibitor of the conversion of isopententyl pyrophosphate (IPP) to farnesyl pyrophosphate (FPP), an inhibitor of the isoprenylation of proteins, an inhibitor of the induction of NF-kβ, an inhibitor of the farnesylation of Ras, an inhibitor of cAMP phosphodiesterase, an antioxidant that blocks LPS- and cytokine-induced production of NO, an enhancer of intracellular levels of cAMP, and any combinations thereof.

18. The method of claim 17, wherein the ratio of the C-peptide level of the patient after treatment compared to the C-peptide level of the patient prior to treatment is at least about 1.4 to 1.0.

19. The method of claim 17, wherein the C-peptide level of the patient after treatment compared to the C-peptide level of the patient prior to treatment increases at least about 3-fold.

20. The method of claim 17, wherein the amount of one or more therapeutic agents administered to the juvenile patient is sufficient to increase the C-peptide level of the patient after at least two years as compared to the C-peptide level of the patient prior to treatment.
